SEQUENCE LISTING (39 sequences)

SEQ ID NO: 1 - protein, 112 aa, Artificial Sequence
  Heavy chain variable region sequence of 2161D2 clone antibody (protein)
QVKLEQSGGGLVQPGGSRKLSCAASGFTFSSFGMHWVRQAPEKGLEWVAYISSGSSTLHYADTVKGRFTISRDNPKNTLFLQMKLPSLCYGLLGSRDLSHRLLSQNDTPICL

SEQ ID NO: 2 - DNA, 336 bp, Artificial Sequence
  Heavy chain variable region sequence of 2161D2 clone antibody (DNA)
caggtcaagctggagcagtctgggggaggcttagtgcagcctggagggtcccggaaactctcctgtgcagcctctggattcactttcagtagctttggaatgcactgggttcgtcaggctccagagaaggggctggagtgggtcgcatacattagtagtggcagtagtaccctccactatgcagacacagtgaagggccgattcaccatctccagagacaatcccaagaacaccctgttcctgcaaatgaaactaccctcactatgctatggactactggggtcaagggacctcagtcaccgtctcctcagccaaaacgacacccccatctgtcta

SEQ ID NO: 3 - protein, 108 aa, Artificial Sequence
  Light chain variable region sequence of 2161D2 clone antibody (Protein)
DIVLTQTNASLAVSLGQRATISYRASKSVSTSGYSYMHWNQQKPGQPPRLLIYLVSNLESGVPVRFSGSGSGTDFTLNIHPVEEEDAATYYCQHIRELTRSEGGPSWK

SEQ ID NO: 4 - DNA, 356 bp, Artificial Sequence
  Light chain variable region sequence of 2161D2 clone antibody (DNA)
gatattgtgctcacccaaactaacgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgtcaggttcagtggcagtgggtctgggacagacttcaccctcaacatccatcctgtggaggaggaggatgctgcaacctattactgtcagcacattagggagcttacacgttcggaggggggaccaagctggaaataaaacgggctgatgctgcaccaactgtatcc

SEQ ID NO: 5 - protein, 112 aa, Artificial Sequence
  Heavy chain variable region sequence of 2164H5 clone antibody (Protein)
EVQLQESGGGLVQPGGSRKLSCAASGFTFSSFGMHWVRQAPEKGLEWVAYISSGSSTLHYADTVKGRFTISRDNPKNTLFLQMKLPSLCYGLLGSRNLSHRLLSQNDTPICL

SEQ ID NO: 6 - DNA, 337 bp, Artificial Sequence
  Heavy chain variable region sequence of 2164H5 clone antibody (DNA)
gaagttcagctgcaggagtctgggggaggcttagtgcagcctggagggtcccggaaactctcctgtgcagcctctggattcactttcagtagctttggaatgcactgggttcgtcaggctccagagaaggggctggagtgggtcgcatacattagtagtggcagtagtaccctccactatgcagacacagtgaagggccgattcaccatctccagagacaatcccaagaacaccctgttcctgcaaatgaaactaccctcactatgctatggactactggggtcaaggaacctcagtcaccgtctcctcagccaaaacgacacccccatctgtctat

SEQ ID NO: 7 - protein, 120 aa, Artificial Sequence
  Light chain variable region sequence of 2164H5 clone antibody (Protein)
DIVITQSNASLAVSLGQRATISSRASESLEYYGISLMHWYLQKPGQPPKLLIYAASNVESGVPARFSGSGSGTDFSLNIHPVEEDDIAMYFCQQSRKVPSTFGGGTKLEIKRADAAPTVS

SEQ ID NO: 8 - DNA, 360 bp, Artificial Sequence
  Light chain variable region sequence of 2164H5 clone antibody (DNA)
gatattgtgatcacccagtctaacgcctctttggctgtgtctctggggcagagagccaccatctccagcagagccagtgaaagtcttgaatattatggcataagtttaatgcactggtacctacagaaaccaggacagccacccaaactcctcatctatgctgcatccaacgtagaatctggggtccctgccaggtttagtggcagtgggtcagggacagacttcagcctcaacatccatcctgtggaggaggatgatattgcaatgtatttctgtcagcaaagtaggaaggttccttcgacgttcggtggaggcaccaagctggaaatcaaacgggctgatgctgcaccaactgtatcc

SEQ ID NO: 9 - protein, 125 aa, Artificial Sequence
  Heavy chain variable region sequence of 2172B12 clone antibody (Protein)
EVQLEQSGGGLVKPGGSLKLSCAASGITFSGYAMSWIRQTPEKRLEWVATISSGGNYTYYPDTVKGRFTISRDNAKNTLYLQVSSLRSEDTAIYYCARLYYGFDYWGQGTTLTVSSAKTTPPSVY

SEQ ID NO: 10 - DNA, 375 bp, Artificial Sequence
  Heavy chain variable region sequence of 2172B12 clone antibody (DNA)
gaagtccagctggagcagtcagggggaggccttgtgaagcctggagggtccctaaaactctcctgtgcagcctctggaattactttcagtggctatgccatgtcttggattcgccagactccggagaagagactggagtgggtcgcaaccattagtagtggtggtaattacacctactatccagacactgtgaaggggcgtttcaccatctccagagacaatgccaaaaacaccctatacttacaagtgagcagtctgaggtctgaggacacggccatatattactgtgcaagactttactacggattcgactactggggccaaggcaccactctcacagtctcctcagccaaaacgacacccccatctgtctat

SEQ ID NO: 11 - protein, 117 aa, Artificial Sequence
  Light chain variable region sequence of 2172B12 clone antibody (Protein)
DIVLTQSNKIMSASVGDRVSVTCKASQNVDSNVAWYQQKPGHSPKALIYSASYRYSRVPDRFTGSGSGTDFTLTITNVQSEDLADYFCQQYHSYPLLTFGAGTKLELKRADAAPTVS

SEQ ID NO: 12 - DNA, 351 bp, Artificial Sequence
  Light chain variable region sequence of 2172B12 clone antibody (DNA)
gacattgtgctcacccaatctaacaaaatcatgtccgcatcagtaggagaccgggtcagtgtcacctgcaaggccagtcagaatgtggatagtaatgtggcctggtatcaacagaaacctggacattctcccaaagcactaatttattcggcatcctaccggtacagtagagtccctgatcgcttcacaggcagtggatctgggacagatttcactctcaccatcaccaatgtgcagtctgaagacttggcagactatttctgtcagcaatatcacagctatcctcttctcacgttcggtgctgggaccaagctggagctgaaacgggctgatgctgcaccaactgtatcc

SEQ ID NO: 13 - protein, 128 aa, Artificial Sequence
  Heavy chain variable region sequence of 2166G4 clone antibody (Protein)
EVQLEESGAELVKPGASVKLSCKASGYSFTSYYMYWVKERPGQGLEWIGGINPTNGNTDFNENFKNKATLTVDKSSSTVYMRLSGLTSEDSAVYYCSRSKYGYAWFAYWGRGTLVTVSAAKTTPPSVY

SEQ ID NO: 14 - DNA, 384 bp, Artificial Sequence
  Heavy chain variable region sequence of 2166G4 clone antibody (DNA)
gaggtccagctggaggagtctggggctgaactggtgaagcctggggcttcagtgaagttgtcctgcaaggcttctggctactccttcaccagctactatatgtactgggtgaaggagaggcctggacaaggccttgagtggattggggggatcaatcctaccaatggtaatactgacttcaatgagaacttcaagaacaaggccacactgactgtagacaaatcctccagcacagtctatatgcgactcagcggcctgacatctgaggactctgcggtctattattgttcaagatctaaatatggttacgcctggtttgcttactggggccgagggactctggtcactgtctctgcagccaaaacgacacccccatctgtctat

SEQ ID NO: 15 - protein, 119 aa, Artificial Sequence
  Light chain variable region sequence of 2166G4 clone antibody (Protein)
DIVLTQSHASLTVSLGQRAAISCRSSQSVNTSAYSYVHSYQQRPGQPPKLLIKYASNLESGVPARFSVSGSGTDFTLNIHSVEEEDPTTYDCQHSWEIHSRSARGQSWKYNGLMLQTVS

SEQ ID NO: 16 - DNA, 357 bp, Artificial Sequence
  Light chain variable region sequence of 2166G4 clone antibody (DNA)
gacattgtgctgacccaatctcacgcttccttaactgtatctctggggcagagggccgccatctcatgcaggtccagccaaagtgtcaatacatctgcctatagttatgtgcactcctaccaacagagaccaggccagccacccaaactcctcatcaagtatgcatccaacctagaatctggggtccctgccaggttcagtgtcagtgggtctgggacagacttcaccctcaacatccattctgtggaggaggaggatcctacaacatatgactgtcagcacagttgggagattcattcacgttcggctcgggggcaaagttggaaatataacgggctgatgctgcaaactgtatcc

SEQ ID NO: 17 - protein, 2322 aa, Artificial Sequence
  NG2/CSPG4 ectodomain
MQSGPRPPLPAPGLALALTLTMLARLASAASFFGENHLEVPVATALTDIDLQLQFSTSQPEALLLLAAGPADHLLLQLYSGRLQVRLVLGQEELRLQTPAETLLSDSIPHTVVLTVVEGWATLSVDGFLNASSAVPGAPLEVPYGLFVGGTGTLGLPYLRGTSRPLRGCLHAATLNGRSLLRPLTPDVHEGCAEEFSASDDVALGFSGPHSLAAFPAWGTQDEGTLEFTLTTQSRQAPLAFQAGGRRGDFIYVDIFEGHLRAVVEKGQGTVLLHNSVPVADGQPHEVSVHINAHRLEISVDQYPTHTSNRGVLSYLEPRGSLLLGGLDAEASRHLQEHRLGLTPEATNASLLGCMEDLSVNGQRRGLREALLTRNMAAGCRLEEEEYEDDAYGHYEAFSTLAPEAWPAMELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVNDPPHIIFPHGSLMVILEHTQKPLGPEVFQAYDPDSACEGLTFQVLGTSSGLPVERRDQPGEPATEFSCRELEAGSLVYVHRGGPAQDLTFRVSDGLQASPPATLKVVAIRPAIQIHRSTGLRLAQGSAMPILPANLSVETNAVGQDVSVLFRVTGALQFGELQKQGAGGVEGAEWWATQAFHQRDVEQGRVRYLSTDPQHHAYDTVENLALEVQVGQEILSNLSFPVTIQRATVWMLRLEPLHTQNTQQETLTTAHLEATLEEAGPSPPTFHYEVVQAPRKGNLQLQGTRLSDGQGFTQDDIQAGRVTYGATARASEAVEDTFRFRVTAPPYFSPLYTFPIHIGGDPDAPVLTNVLLVVPEGGEGVLSADHLFVKSLNSASYLYEVMERPRHGRLAWRGTQDKTTMVTSFTNEDLLRGRLVYQHDDSETTEDDIPFVATRQGESSGDMAWEEVRGVFRVAIQPVNDHAPVQTISRIFHVARGGRRLLTTDDVAFSDADSGFADAQLVLTRKDLLFGSIVAVDEPTRPIYRFTQEDLRKRRVLFVHSGADRGWIQLQVSDGQHQATALLEVQASEPYLRVANGSSLVVPQGGQGTIDTAVLHLDTNLDIRSGDEVHYHVTAGPRWGQLVRAGQPATAFSQQDLLDGAVLYSHNGSLSPRDTMAFSVEAGPVHTDATLQVTIALEGPLAPLKLVRHKKIYVFQGEAAEIRRDQLEAAQEAVPPADIVFSVKSPPSAGYLVMVSRGALADEPPSLDPVQSFSQEAVDTGRVLYLHSRPEAWSDAFSLDVASGLGAPLEGVLVELEVLPAAIPLEAQNFSVPEGGSLTLAPPLLRVSGPYFPTLLGLSLQVLEPPQHGALQKEDGPQARTLSAFSWRMVEEQLIRYVHDGSETLTDSFVLMANASEMDRQSHPVAFTVTVLPVNDQPPILTTNTGLQMWEGATAPIPAEALRSTDGDSGSEDLVYTIEQPSNGRVVLRGAPGTEVRSFTQAQLDGGLVLFSHRGTLDGGFRFRLSDGEHTSPGHFFRVTAQKQVLLSLKGSQTLTVCPGSVQPLSSQTLRASSSAGTDPQLLLYRVVRGPQLGRLFHAQQDSTGEALVNFTQAEVYAGNILYEHEMPPEPFWEAHDTLELQLSSPPARDVAATLAVAVSFEAACPQRPSHLWKNKGLWVPEGQRARITVAALDASNLLASVPSPQRSEHDVLFQVTQFPSRGQLLVSEEPLHAGQPHFLQSQLAAGQLVYAHGGGGTQQDGFHFRAHLQGPAGASVAGPQTSEAFAITVRDVNERPPQPQASVPLRLTRGSRAPISRAQLSVVDPDSAPGEIEYEVQRAPHNGFLSLVGGGLGPVTRFTQADVDSGRLAFVANGSSVAGIFQLSMSDGASPPLPMSLAVDILPSAIEVQLRAPLEVPQALGRSSLSQQQLRVVSDREEPEAAYRLIQGPQYGHLLVGGRPTSAFSQFQIDQGEVVFAFTNFSSSHDHFRVLALARGVNASAVVNVTVRALLHVWAGGPWPQGATLRLDPTVLDAGELANRTGSVPRFRLLEGPRHGRVVRVPRARTEPGGSQLVEQFTQQDLEDGRLGLEVGRPEGRAPGPAGDSLTLELWAQGVPPAVASLDFATEPYNAARPYSVALLSVPEAARTEAGKPESSTPTGEPGPMASSPEPAVAKGGFLSFLEANMFSVIIPMCLVLLLLALILPLLFYLRKRNKTGKHDVQVLTAKPRNGLAGDTETFRKVEPGQAIPLTAVPGQGPPPGGQPDPELLQFCRTPNPALKNGQYWV

SEQ ID NO: 18 - DNA, 24 bp, Artificial Sequence
  CDR1 Heavy chain variable region 2161D2
ggattcactttcagtagctttgga

SEQ ID NO: 19 - DNA, 24 bp, Artificial Sequence
  CDR2 Heavy chain variable region 2161D2
attagtagtggcagtagtaccctc

SEQ ID NO: 20 - DNA, 30 bp, Artificial Sequence
  CDR1 Light chain variable region 2161D2
aaaagtgtcagtacatctggctatagttat

SEQ ID NO: 21 - DNA, 9 bp, Artificial Sequence
  CDR2 Light chain variable region 2161D2
cttgtatcc

SEQ ID NO: 22 - DNA, 24 bp, Artificial Sequence
  CDR3 Light chain variable region 2161D2
cagcacattagggagcttacacgt

SEQ ID NO: 23 - DNA, 24 bp, Artificial Sequence
  CDR1 Heavy chain variable region 2164H5
ggattcactttcagtagctttgga

SEQ ID NO: 24 - DNA, 24 bp, Artificial Sequence
  CDR2 Heavy chain variable region 2164H5
attagtagtggcagtagtaccctc

SEQ ID NO: 25 - DNA, 30 bp, Artificial Sequence
  CDR1 Light chain variable region 2164H5
gaaagtcttgaatattatggcataagttta

SEQ ID NO: 26 - DNA, 9 bp, Artificial Sequence
  CDR2 Light chain variable region 2164H5
gctgcatcc

SEQ ID NO: 27 - DNA, 27 bp, Artificial Sequence
  CDR3 Light chain variable region 2164H5
cagcaaagtaggaaggttccttcgacg

SEQ ID NO: 28 - DNA, 24 bp, Artificial Sequence
  CDR1 Heavy chain variable region 2166G4
ggctactccttcaccagctactat

SEQ ID NO: 29 - DNA, 24 bp, Artificial Sequence
  CDR2 Heavy chain variable region 2166G4
atcaatcctaccaatggtaatact

SEQ ID NO: 30 - DNA, 27 bp, Artificial Sequence
  CDR3 Heavy chain variable region 2166G4
tcaagatctaaatatggttacgcctgg

SEQ ID NO: 31 - DNA, 30 bp, Artificial Sequence
  CDR1 Light chain variable region 2166G4
caaagtgtcaatacatctgcctatagttat

SEQ ID NO: 32 - DNA, 9 bp, Artificial Sequence
  CDR2 Light chain variable region 2166G4
tatgcatcc

SEQ ID NO: 33 - DNA, 27 bp, Artificial Sequence
  CDR3 Light chain variable region 2166G4
cagcacagttgggagattcattcacgt

SEQ ID NO: 34 - DNA, 24 bp, Artificial Sequence
  CDR1 Heavy chain variable region 2172B12
ggaattactttcagtggctatgcc

SEQ ID NO: 35 - DNA, 24 bp, Artificial Sequence
  CDR2 Heavy chain variable region 2172B12
attagtagtggtggtaattacacc

SEQ ID NO: 36 - DNA, 27 bp, Artificial Sequence
  CDR3 Heavy chain variable region 2172B12
gcaagactttactacggattcgactac

SEQ ID NO: 37 - DNA, 18 bp, Artificial Sequence
  CDR1 Light chain variable region 2172B12
cagaatgtggatagtaat

SEQ ID NO: 38 - DNA, 9 bp, Artificial Sequence
  CDR2 Light chain variable region 2172B12
tcggcatcc

SEQ ID NO: 39 - DNA, 30 bp, Artificial Sequence
  CDR3 Light chain variable region 2172B12
cagcaatatcacagctatcctcttctcacg

(19) United States
(12) Patent Application Publication
(10) Pub. No.: US 2017/0369588 A1
(43) Pub. Date: Dec. 28, 2017

(54) PRO-APOPTOTIC ANTI-NG2/CSPG4 ANTIBODIES AND THEIR USES FOR DISEASE THERAPY

(71) Applicant: UNIVERSITA' DEGLI STUDI DI PARMA, Parma (IT)

(72) Inventors: Roberto Perris, Milano (IT); Alice Dallatomasina, Milano (IT); Pier Andrea Nicolosi, Cordenos (Pordenone) (IT); Elisa Tamburini, Parma (IT); Maria Teresa Mucignat, Aviano (Pordenone) (IT)

(73) Assignee: UNIVERSITA' DEGLI STUDI DI PARMA, Parma (IT)

(21) Appl. No.: 15/538,659

(22) PCT Filed: Dec. 23, 2015
(86) PCT No.: PCT/IB2015/059921

§ 371 Date: Jun. 22, 2017

Related U.S. Application Data
(60) Provisional application No. 62/095,836, filed on Dec. 23, 2014.

(30) Foreign Application Priority Data
Dec. 23, 2014 (IT) MI2014A002225

(51) Int. Cl. C07K 16/30; G01N 33/574; A61K 39/395; A61K 45/06

(52) U.S. Cl. CPC C07K 16/3053; A61K 45/06; G01N 33/574; A61K 39/395; C07K 2317/33; C07K 2317/76; C07K 2317/73

(57) ABSTRACT

The present invention relates to an antibody capable of binding with high-affinity and high selectivity to the ectodomain of the transmembrane proteoglycan (PG) NG2/CSPG4, preferably to discrete isoforms of said PG, preferably isoforms that may be generated by alternative splicing, and/or coding single nucleotide polymorphisms, and/or post-transcriptional and/or post-translational modifications. The invention further relates to an anti-NG2/CSPG4 antibody possessing the ability to uniquely induce programmed cell death, exhibited as both canonical caspase-dependent apoptosis and authophagy, in NG2/CSPG4-expressing cancer cells. This action being manifested irrespectively of the coaction of other exogenously added factors. Moreover, the present invention refers to a composition comprising the antibody of the invention, in its naked, encapsulated or genetically engineered form, as pharmaceutical excipient. A further aspect of the present invention refers to the anti-NG2/CSPG4 molecule, or any of its isoforms and fragments, provided as proteolytically generated peptides or produced synthetically and/or recombinantly, for the treatment of apoptosis and/or autophagy-dependent diseases, including but not restricted to cancer.

The present invention relates to an antibody capable of binding with high-affinity and high selectivity to the ectodomain of the transmembrane proteoglycan (PG) NG2/CSPG4. The invention specifically refers to said antibody possessing the ability to uniquely induce programmed cell death through canonical caspase-dependent apoptosis or authophagy, in cancer cells specifically expressing such protein, and without the participation of other exogenously added factors. Moreover, the present invention refers to a composition comprising the antibody of the invention as pharmaceutical excipient.

A further aspect of the present invention refers to the anti-NG2/CSPG4 molecule, or any of its isoforms and fragments, provided as proteolytically generated peptides or produced synthetically and/or recombinantly, for the treatment of apoptosis and/or autophagy-dependent pathologies, including but not restricted to cancer and related diseases.

### STATE OF THE ART

Nerve/Glia Antigen 2 (NG2) is a transmembrane proteoglycan (PG) governing the interaction of cancer cells with their microenvironment. Moreover, NG2 promotes the myriad of cellular events prompting tumor growth and cancer cell spreading. NG2 is also known as High Molecular Weight Melanoma-Associated Antigen (HMW-MAA), or simply Melanoma Cell Surface Proteoglycan (MCSP/MCSPG) because it was originally identified also on cells derived from cutaneous melanoma lesions. The human NG2 orthologous gene is coded Chondroitin Sulfate ProteoGlycan-4 (CSPG4) and it is located on chromosome 15:24q2.

NG2/CSPG4 interacts with several molecules, such as growth factors, signaling molecules, metalloproteinases and a various ECM components through its extended extracellular domain, which may associate with and control intracellularly the cell surface receptors for these ligands. Through these interactions, the PG acts a primary promoter of cell survival and apoptotic resistance, a function that is underpinned by its ability to activate the PI-3K-Akt-mTOR signaling pathways upon PCKα-induced phosphorylation of one of its cytoplasmic threonine residues (see below). In fact, cancer cells exhibiting high surface expression show enhanced chemoresistance. Intriguingly, in certain cultivated cellular models, forced overexpression of NG2/CSPG4, possibly along with downregulation of a4 integrins, may enhance anoikis and this effect is curtailed by MMP13-operated shedding of the NG2/CSPG4 ectodomain. However, evidence for such a mechanism on cells constitutively expressing high levels of NG2/CSPG4 has not been provided, nor documented to be operating in vivo.

In highly motile cells, such as cancer cells, NG2/CSPG4 accumulates in the advancing cytoplasmic front, preferentially in filopodial extensions and at cell-substrate contact areas. The cytoplasmic tail of NG2/CSPG4 contains two threonine residues undergoing differential phosphorylation by PKCα (Thr2256) and ERK(s) (Thr2314) depending upon the cellular events in which the PG may participate.

NG2/CSPG4 has not been identified as a factor directly promoting tumor formation; however, it has been shown to become de novo expressed in a number of solid and hematological neoplastic conditions, as well as to be linked to cancer-associated epithelial-mesenchymal transitions. The most striking examples of this latter transitional linkage are the appearance of NG2/CSPG4 on melanoma cells, breast, head and neck carcinomas and mesotheliomas.

Even though high transcriptional levels of the CSPG4 gene may be detected, appearance of PG on the surface of cancer cells may not always occur (i.e. the PG is primary detected intracellularly) and is likely to be a mere secondary event of tumor formation.

At present, augmented transcriptional and/or translational levels of NG2/CSPG4 have currently been disclosed in more than 30 solid tumor types (and their subvariants), and, in several of them, a certain diagnostic and/or prognostic connotation of the PG has been proposed.

This would suggest that the PG drives metastasis formation, as proposed by some experimental data in mouse models.

Furthermore, although cells engineered to overexpress NG2/CSPG4 are generally more malignant than their counterpart NG2/CSPG4-deficient cells, unequivocal experimental proof of a direct metastasis-promoting effect of the PG is currently missing.

Given its documented diagnostic impact, it appears perplexing that NG2/CSPG4 has remained largely unutilized as a biomarker for the routine clinical monitoring of cancer patients. This may be due to the failure of most of the published studies to demonstrate the independence of dysregulated NG2/CSPG4 expression from established clinical parameters.

Several in vitro assays support the concept that NG2/CSPG4 may affect drug sensitivity of cancer cells by reinforcing their interactions with the host microenvironment, especially with the stromal ECM, by modulating the activity of certain integrins and, through these actions, by activating pro-survival signaling cascades.

### SUMMARY OF THE INVENTION

A first aspect of the present invention refers to an antibody, preferably a monoclonal antibody, recognizing the extracellular domain of NG2/CSPG4 and its ability to induce apoptosis and/or autophagy upon antigen-binding in cancer cells expressing NG2/CSPG4.

Therefore, the antibody of the invention can be used for cell growth inhibition by exposing a cell expressing NG2/CSPG4 to a therapeutic amount of an antibody capable of binding to NG2/CSPG4.

A second aspect of the present invention refers to a pharmacological composition comprising a therapeutic amount of the antibody, in its entirety or single chain variable fragment (scFv) of the invention that is active against NG2/CSPG4, or any other portion of the antibody, preferably, embodied in a bispecific antibody construct (e.g. an antibody binding both NG2/CSPG4 and another cell surface molecule and/or antigen) or a fusion protein comprised of a therapeutic amount of the antibody, in its entirety or single chain variable fragment, or scFv, an another therapeutic molecule, such as a cytochine or an aptoptosis-inducing agent (e.g TRAIL or Fas Ligand), or a CAR T (Chimeric Antigen Receptor T cell) construct in which the scFv derived from the portion of the antibody of the invention is expressed on the surface of a CAR T cell, such as to simultaneously engaging NG2/CSPG4 expressing cells and endogenous T cells and favor the maintenance of the CAR T cell in proximity to the cancer cell to trigger the activation of the engaged T cell.

Further provided by the invention is the use of the antibody and the composition of the invention for selective trigger of apoptosis and/or autophagy in cells specifically expressing NG2/CSPG4 recognized by the antibody.

A further aspect of present invention refers to the antibody and the composition of the invention for use in the treatment of an apoptosis and/or autophagy-dependent disease, preferably cancer, more preferably solid or hematological cancers, as well as cancer-related diseases.

The present invention further relates to nucleic acid sequences or amino acid sequences encoding the heavy and light chain immunoglobulins or the complementarity determining regions of the antibody.

The present invention provides also a host cell producing the antibody and its variants. A further aspect of the present invention refers to a naked polypeptide formulation of the naturally produced and/or humanized and/or genetically engineered and/or recombinantly produced and/or synthetically derived compound active in inducing cell death and including at least one of the anti-NG2/CSPG4 antibody, or fragments thereof, of the invention.

### DESCRIPTION OF THE DRAWINGS

FIG. 1 shows the identification of NG2/CSPG4 isoforms and the proteolytic fragments in cells (1-A-A375 and HT1080^{NG2+} cells) and in tissues (1-B pericyte sprouts of fetal brain neovessels and in glioblastoma multiforme (GMB) lesions. Recombinant NG2/CSPG4 (NG2^{rec}) is included as a control.

FIG. 2 shows the effects on cell migration of the antibodies of the invention (hybridoma clones 2172B12/C10, 2166G4/C10/D1, 2164H5/E11, 2161D2/C2 and their subclonal derivations). The cell migration model used is the wound-healing scratch assay performed using HT1080^{NG2+}as model cell line.

FIG. 3 (I-II) shows the immunostaining of HT1080^{NG2+}, A375 and HS578T cancer cells and primary human brain vascular pericytes (HBVP) by using the antibodies of the invention (hybridoma clones 2172B12/C10, 2166G4/C10/D1, 2164H5/E11,2161D2/C2 and their subclonal derivations).

FIGS. 4 shows the effect of the antibodies of the invention (hybridoma clones 2164H5/E11, 2161 D2/C2 and their subclonal derivations) on cultured tumor cells (A375, HT1080^{NG2+} and HS578) in monolayered (2D-A) or as multicellular spheroidal configurations (3D-B,C).

FIG. 5 shows advanced apoptosis, measured by using the TUNEL assay, in A375 cells (A) and HT1080^{NG2+} cells (B) and induced by administration of the antibodies of the invention (hybrdioma clones 2172B12/C10, 2166G4/C10/D1, 2164H5/E11 and 2161 D2/C2 and their subclone variants) to the cells.

FIG. 6 shows apoptosis measured by multiparametric assay induced by administering the antibodies of the invention (hybridoma clones 2172B12/C10, 2166G4/C10/D1, 2164H5/E11, 2161 D2/C2 and their subclonal derivations) on A375, HT1080^{NG2+} cell lines.

FIG. 7 shows the PARP cleavage detection by using flow cytometry on the following cancer cell lines: A375 (A), HT1080^{NG2+} (B) and HS578 (C) after administering the antibodies of the invention (hybridoma clones 2172B12/C10, 2166G4/C10/D1, 2164H5/E11, 2161 D2/C2 and their subclonal derivations).

FIG. 8 shows activation of caspases responsible for propagation of the extrinsic apoptotic pathway after administration of the antibodies of the invention (hybridoma clones 2172B12/C10, 2166G4/C10/D1, 2164H5/E11, 2161D2/C2 and their subclonal derivations) in 2D and 3D culture of the model cancer cell lines (A375, HT1080^{NG2+} and HT578T).

FIG. 9 shows autophagy detection in the A375 cancer cell line transiently transfected with a LC3-GFP fusion construct and treated with antibodies of the invention by using the LC3-aggregation method.

### DETAILED DESCRIPTION OF THE INVENTION

In the contest of the present invention, the term “apoptosis” means the process of programmed cell death (PCD) that may occur in multicellular organisms. Biochemical events lead to characteristic cell changes (morphology) and death. These changes include for example, blebbing, cell shrinkage, nuclear fragmentation, chromatin condensation, or chromosomal DNA fragmentation. Excessive apoptosis causes atrophy, whereas an insufficient amount results in uncontrolled cell proliferation, such as cancer.

Many pathways and signals lead to apoptosis. After a cell receives stimulus, it undergoes organized degradation of cellular organelles by activated proteolytic caspases. A cell undergoing apoptosis shows the following characteristic morphology:

Cell shrinkage and rounding are shown because of the breakdown of the proteinaceous cytoskeleton by caspases.

The cytoplasm appears dense, and the organelles appear tightly packed.

Chromatin undergoes condensation into compact patches against the nuclear envelope in a process known as pyknosis, a hallmark of apoptosis.

The nuclear envelope becomes discontinuous and the DNA inside it is fragmented in a process referred to as karyorrhexis. The nucleus breaks into several discrete chromatin bodies or nucleosomal units due to the degradation of DNA.

The cell membrane shows irregular buds known as blebs.

The cell breaks apart into several vesicles called apoptotic bodies, which are then phagocytosed.

Caspases play the central role in the transduction of Death Receptor (DR) apoptotic signals.

Caspases are proteins that are highly conserved, cysteine-dependent aspartate-specific proteases. There are two types of caspases: initiator caspases and effector caspases. The activation of initiator caspases requires binding to specific oligomeric activator protein. Effector caspases are then activated by these active initiator caspases through proteolytic cleavage. The active effector caspases then proteolytically degrade a host of intracellular proteins to carry out the cell death program.

Therefore, in a preferred embodiment the apoptosis of the present invention is caspase-dependent.

There also exists a caspase-independent apoptotic pathway that is mediated by AIF (apoptosis-inducing factor).

In the contest of the present invention, the term “autophagy” (or autophagocytosis) means the basic catabolic mechanism that involves cell degradation of unnecessary or dysfunctional cellular components through the actions of lysosomes.

The breakdown of cellular components promotes cellular survival during starvation by maintaining cellular energy levels. Autophagy allows the degradation and recycling of cellular components. During this process, targeted cytoplasmic constituents are isolated from the rest of the cell within a double-membrane vesicle known as an autophagosome. The autophagosome then fuses with a lysosome and its cargo is degraded and recycled. There are three different forms of autophagy that are commonly described: macroautophagy, microautophagy and chaperone-mediated autophagy.

In the context of disease, autophagy has been seen as an adaptive response to stress promoting survival, whereas in other cases it appears to promote cell death and morbidity.

One of the mechanisms of programmed cell death (PCD) is associated with the appearance of autophagosomes and depends on autophagy proteins. This form of cell death most likely corresponds to a process that has been morphologically defined as autophagic PCD.

A first aspect of the present invention refers to an antibody able to recognize and bind to the ectodomain of the NG2/CSPG4 transmembrane proteoglycan. Applicants have found that the antibody induces cancer cell apoptosis and/or autophagy upon high-affinity binding to its antigen. In particular, the antibody of the invention recognizes and bind with high-affinity and high selectivity discrete NG2/CSPG4 isoforms or the proteolitically processed forms of the protein.

The NG2/CSPG4 isoform, or the proteolitically processed form of the protein to which the present invention refers to has a molecular weight ranging from 50-280 kDa, preferably from 110-260 kDa, more preferably from 120 to 250 kDa.

Therefore, the present invention refers to an apoptosis and/or autophagy inducing antibody able to recognize and bind with high-affinity and high selectivity NG2/CSPG4, in particular discrete NG2/CSPG4 isoforms. In other words, after binding the ectodomain of the cognate NG2/CSPG4 antigen expressed on cancer cells, the antibody of the invention activates at least one of the pathways above known to cause and/or be involved in the execution of programmed cell death.

The antibody of the invention is preferably a monoclonal antibody.

The antibody of the invention recognizes and binds with high-affinity a defined portion of the ectodomain of NG2/CSPG4, preferably present in discrete NG2/CSPG4 isoforms, said ectodomain of NG2/CSPG4 being preferably SEQ ID NO: 17, corresponding to the NCBI Reference Sequence NC 000015.10.

As already stated, NG2 is the orthologous gene of human CSPG4 in rat. NG2/CSPG4 is expressed on cancer cells and angiogenic vasculature, and its expression is associated with an aggressive disease course in several malignancies. Alternative names of NG2/CSPG4 are High Molecular Weight Melanoma-Associated Antigen (HMW-MAA), Melanoma Cell Surface Proteoglycan (MCSP, MCSPG) and melanoma proteoglycan (Mel-PG).

Preferably, Human CSPG4 gene corresponds to the NCBI Reference Sequence: NM001897.4.

Preferably, NG2 gene corresponds to the NCBI Reference Sequence: NM_031022.1. According to a preferred embodiment of the present invention the antibody is characterized by:

SEQ ID NO: 18, 19 and SEQ ID NO: 20, 21, 22, wherein SEQ ID NO: 18, 19 are respectively CDR1 and CDR2 of the heavy chain of the antibody, and SEQ ID NO: 20, 21, 22 are respectively CDR1, CDR2 and CDR3 of the antibody, wherein the antibody is preferably produced by the hybridoma cell line 2161 D2; and/or

SEQ ID NO: 23, 24 and SEQ ID NO: 25, 26, 27, wherein SEQ ID NO: 23, 24 are respectively CDR1 and CDR2 of the heavy chain of the antibody, and SEQ ID NO: 25, 26, 27 are respectively CDR1, CDR2 and CDR3 of the antibody, wherein the antibody is preferably produced by the hybridoma cell line 2164H5; and/or

SEQ ID NO: 28, 29, 30 and SEQ ID NO: 31, 32, 33, wherein SEQ ID NO: 28, 29, 30 are respectively CD R1, CDR2 and CDR3 of the heavy chain of the antibody, and SEQ ID NO: 31, 32, 33 are respectively CDR1, CDR2 and CDR3 of the antibody, wherein the antibody is preferably produced by the hybridoma cell line 2166G4; and/or

SEQ ID NO: 34, 35, 36 and SEQ ID NO: 37, 38, 39, wherein SEQ ID NO: 34, 35, 36 are respectively CD R1, CDR2 and CDR3 of the heavy chain of the antibody, and SEQ ID NO: 37, 38, 39 are respectively CDR1, CDR2 and CDR3 of the antibody, wherein the antibody is preferably produced by the hybridoma cell line 2172612.

According to a preferred embodiment of the present invention the antibody has a variable heavy chain (VH) selected from the group consisting of: SEQ ID NO: 1, 5, 9 and 13 or the corresponding nucleic sequences SEQ ID NO: 2, 6, 10 and 14.

According to a preferred embodiment of the present invention the antibody has a variable light chain (LH) selected from the group consisting of: SEQ ID: 3, 7, 11 and 15 or the corresponding nucleic sequences SEQ ID NO: 4, 8, 12 and 16.

According to a preferred embodiment the antibody has a VH selected from the group consisting of: SEQ ID NO: 1, 5, 9 and 13 or the corresponding nucleic sequences SEQ ID NO: 2, 6, 10 and 14 and a LH selected from the group consisting of: SEQ ID: 3, 7, 11 and 15 or the corresponding nucleic sequences SEQ ID NO: 4, 8, 12 and 16.

According to a preferred embodiment of the present invention, the antibody has SEQ ID NO: 1 as VH and SEQ ID NO: 3 as VL or the corresponding nucleic sequence SEQ ID NO: 2 and 4.

According to a preferred embodiment of the present invention, the antibody has SEQ ID NO: 5 as VH and SEQ ID NO: 7 as VL or the corresponding nucleic sequence SEQ ID NO: 6 and 8.

According to a preferred embodiment of the present invention, the antibody has SEQ ID NO: 9 as VH and SEQ ID NO: 11 as VL or the corresponding nucleic sequence SEQ ID NO: 10 and 12.

According to a preferred embodiment of the present invention, the antibody has SEQ ID NO: 13 as VH and SEQ ID NO: 15 as VL or the corresponding nucleic sequence SEQ ID NO: 14 and 16.

In a preferred form of the invention, the monoclonal antibody has preferably the VH and VL of the murine hybridoma cell line deposited at International Depositary Authority of Canada with the Accession Number 121214-01.

In a further preferred form of the invention, the monoclonal antibody has preferably the VH and VL of the murine hybridoma cell line deposited at International Depositary Authority of Canada with the Accession Number 121214-02.

In a further preferred form of the invention, the monoclonal antibody has preferably the VH and VL of the murine hybridoma cell line deposited at International Depositary Authority of Canada with the Accession Number 121214-03.

In a further preferred form of the invention, the monoclonal antibody has preferably the VH and VL of the murine hybridoma cell line deposited at International Depositary Authority of Canada with the Accession Number 121214-04.

The hybridoma having the Accession Number 121214-01 produces the antibody here disclosed as clone 2166G4/C10/D1 (Clone 2166G4).

Preferably, the antibody 2166G4/C10/D1 has SEQ ID NO: 13 as VH and SEQ ID NO: 15 as VL or the corresponding nucleic sequence SEQ ID NO: 14 and 16.

The hybridoma having the Accession Number 121214-02 produces the antibody here disclosed as clone 2172B12/C10 (Clone 2172612).

Preferably, the antibody 2172B12/C10 has SEQ ID NO: 9 as VH and SEQ ID NO: 11 as VL or the corresponding nucleic sequence SEQ ID NO: 10 and 12.

The hybridoma having the Accession Number 121214-03 produces the antibody here disclosed as clone 2164H5/E11 (clone 2164H5).

Preferably, the antibody 2164H5/E11 has SEQ ID NO: 5 as VH and SEQ ID NO: 7 as VL or the corresponding nucleic sequence SEQ ID NO: 6 and 8.

The hybridoma having the Accession Number 121214-04 produces the antibody here disclosed as clone 2161 D2/C2 (clone 2161 D2).

Preferably, the antibody 2161 D2/C2 has SEQ ID NO: 1 as VH and SEQ ID NO: 3 as VL or the corresponding nucleic sequence SEQ ID NO: 2 and 4.

The sequences relevant for the scope of the present invention are summarized in Table I. The sequences of the invention are disclosed according to the international standard WIPO ST.25 and the description thereof was developed by using the program Patent-In 3.5.

Sequence description is reported as follows.

Table I shows all the amino acid and nucleotide sequences and the corresponding “Sequence Identifiers” defining the antibodies disclosed in the present invention.

The subject matter of the present invention also includes all the nucleic sequences derived from the nucleotide sequences shown in Table I because of the genetic code degeneration.

Sequences having 90, 80 or 70 identity percentage have to be considered comprised in the subject matter disclosed in the present invention.

TABLE I

| | | |
|---|---|---|
| Heavy chain | QVKLEQSGGGLVQPGGSRKLSCAASGFTFSSFGMHWVRQAPEKGLEWVAYISSGSSTLHYAD | SEQ ID NO: |
| variable region | TVKGRFTISRDNPKNTLFLQMKLPSLCYGLLGSRDLSHRLLSQNDTPICL | 1 |
| sequence of | | |
| 2161D2 clone | | |
| antibody | | |
| (Protein) | | |
| Heavy chain | CAGGTCAAGCTGGAGCAGTCTGGGGGAGGCTTAGTGCAGCCTGGAGGGTCCCGGAAACTCTC | SEQ ID NO: |
| variable region | CTGTGCAGCCTCTGGATTCACTTTCAGTAGCTTTGGAATGCACTGGGTTCGTCAGGCTCCAG | 2 |
| sequence of | AGAAGGGGCTGGAGTGGGTCGCATACATTAGTAGTGGCAGTAGTACCCTCCACTATGCAGAC | |
| 2161D2 clone | ACAGTGAAGGGCCGATTCACCATCTCCAGAGACAATCCCAAGAACACCCTGTTCCTGCAAAT | |
| antibody (DNA) | GAAACTACCCTCACTATGCTATGGACTACTGGGGTCAAGGGACCTCAGTCACCGTCTCCTCA | |
| | GCCAAAACGACACCCCCATCTGTCTA | |
| Light chain | DIVLTQTNASLAVSLGQRATISYRASKSVSTSGYSYMHWNQQKPGQPPRLLIYLVSNLESGV | SEQ ID NO: |
| variable region | PVRFSGSGSGTDFTLNIHPVEEEDAATYYCQHIRELTRSEGGPSWK. | 3 |
| sequence of | | |
| 2161D2 clone | | |
| antibody | | |
| (Protein) | | |
| Light chain | GATATTGTGCTCACCCAAACTAACGCTTCCTTAGCTGTATCTCTGGGGCAGAGGGCCACCAT | SEQ ID NO: |
| variable region | CTCATACAGGGCCAGCAAAAGTGTCAGTACATCTGGCTATAGTTATATGCACTGGAACCAAC | 4 |
| sequence of | AGAAACCAGGACAGCCACCCAGACTCCTCATCTATCTTGTATCCAACCTAGAATCTGGGGTC | |
| 2161D2 clone | CCTGTCAGGTTCAGTGGCAGTGGGTCTGGGACAGACTTCACCCTCAACATCCATCCTGTGGA | |
| antibody (DNA) | GGAGGAGGATGCTGCAACCTATTACTGTCAGCACATTAGGGAGCTTACACGTTCGGAGGGGG | |
| | GACCAAGCTGGAAATAAAACGGGCTGATGCTGCACCAACTGTATCC | |
| Heavy chain | EVQLQESGGGLVQPGGSRKLSCAASGFTFSSFGMHWVRQAPEKGLEWVAYISSGSSTLHYAD | SEQ ID NO: |
| variable region | TVKGRFTISRDNPKNTLFLQMKLPSLCYGLLGSRNLSHRLLSQNDTPICL | 5 |
| sequence of | | |
| 2164H5 clone | | |
| antibody | | |
| (Protein) | | |
| Heavy chain | GAAGTTCAGCTGCAGGAGTCTGGGGGAGGCTTAGTGCAGCCTGGAGGGTCCCGGAAACTCTC | SEQ ID NO: |
| variable region | CTGTGCAGCCTCTGGATTCACTTTCAGTAGCTTTGGAATGCACTGGGTTCGTCAGGCTCCAG | 6 |
| sequence of | AGAAGGGGCTGGAGTGGGTCGCATACATTAGTAGTGGCAGTAGTACCCTCCACTATGCAGAC | |
| 2164H5 clone | ACAGTGAAGGGCCGATTCACCATCTCCAGAGACAATCCCAAGAACACCCTGTTCCTGCAAAT | |
| antibody (DNA) | GAAACTACCCTCACTATGCTATGGACTACTGGGGTCAAGGAACCTCAGTCACCGTCTCCTCA | |
| | GCCAAAACGACACCCCCATCTGTCTAT | |
| Light chain | DIVITQSNASLAVSLGQRATISSRASESLEYYGISLMHWYLQKPGQPPKLLIYAASNVESGV | SEQ ID NO: |
| variable region | PARFSGSGSGTDFSLNIHPVEEDDIAMYFCQQSRKVPSTFGGGTKLEIKRADAAPTVS | 7 |
| sequence of | | |
| 2164H5 clone | | |
| antibody | | |
| (Protein) | | |
| Light chain | GATATTGTGATCACCCAGTCTAACGCCTCTTTGGCTGTGTCTCTGGGGCAGAGAGCCACCAT | SEQ ID NO: |
| variable region | CTCCAGCAGAGCCAGTGAAAGTCTTGAATATTATGGCATAAGTTTAATGCACTGGTACCTAC | 8 |
| sequence of | AGAAACCAGGACAGCCACCCAAACTCCTCATCTATGCTGCATCCAACGTAGAATCTGGGGTC | |
| 2164H5 clone | CCTGCCAGGTTTAGTGGCAGTGGGTCAGGGACAGACTTCAGCCTCAACATCCATCCTGTGGA | |
| antibody (DNA) | GGAGGATGATATTGCAATGTATTTCTGTCAGCAAAGTAGGAAGGTTCCTTCGACGTTCGGTG | |
| | GAGGCACCAAGCTGGAAATCAAACGGGCTGATGCTGCACCAACTGTATCC | |
| Heavy chain | EVQLEQSGGGLVKPGGSLKLSCAASGITFSGYAMSWIRQTPEKRLEWVATISSGGNYTYYPD | SEQ ID NO: |
| variable region | TVKGRFTISRDNAKNTLYLQVSSLRSEDTAIYYCARLYYGFDYWGQGTTLTVSSAKTTPPSV | 9 |
| sequence of | Y | |
| 2172B12 clone | | |
| antibody | | |
| (Protein) | | |
| Heavy chain | GAAGTCCAGCTGGAGCAGTCAGGGGGAGGCCTTGTGAAGCCTGGAGGGTCCCTAAAACTCTC | SEQ ID NO: |
| variable region | CTGTGCAGCCTCTGGAATTACTTTCAGTGGCTATGCCATGTCTTGGATTCGCCAGACTCCGG | 10 |
| sequence of | AGAAGAGACTGGAGTGGGTCGCAACCATTAGTAGTGGTGGTAATTACACCTACTATCCAGAC | |
| 2172B12 clone | ACTGTGAAGGGGCGTTTCACCATCTCCAGAGACAATGCCAAAAACACCCTATACTTACAAGT | |
| antibody (DNA) | GAGCAGTCTGAGGTCTGAGGACACGGCCATATATTACTGTGCAAGACTTTACTACGGATTCG | |
| | ACTACTGGGGCCAAGGCACCACTCTCACAGTCTCCTCAGCCAAAACGACACCCCCATCTGTC | |
| | TAT | |
| Light chain | DIVLTQSNKIMSASVGDRVSVTCKASQNVDSNVAWYQQKPGHSPKALIYSASYRYSRVPDRF | SEQ ID NO: |
| variable region | TGSGSGTDFTLTITNVQSEDLADYFCQQYHSYPLLTFGAGTKLELKRADAAPTVS | 11 |
| sequence of | | |
| 2172B12 clone | | |
| antibody | | |
| (Protein) | | |
| Light chain | GACATTGTGCTCACCCAATCTAACAAAATCATGTCCGCATCAGTAGGAGACCGGGTCAGTGT | SEQ ID NO: |
| variable region | CACCTGCAAGGCCAGTCAGAATGTGGATAGTAATGTGGCCTGGTATCAACAGAAACCTGGAC | 12 |
| sequence of | ATTCTCCCAAAGCACTAATTTATTCGGCATCCTACCGGTACAGTAGAGTCCCTGATCGCTTC | |
| 2172B12 clone | ACAGGCAGTGGATCTGGGACAGATTTCACTCTCACCATCACCAATGTGCAGTCTGAAGACTT | |
| antibody (DNA) | GGCAGACTATTTCTGTCAGCAATATCACAGCTATCCTCTTCTCACGTTCGGTGCTGGGACCA | |
| | AGCTGGAGCTGAAACGGGCTGATGCTGCACCAACTGTATCC | |
| Heavy chain | EVQLEESGAELVKPGASVKLSCKASGYSFTSYYMYWVKERPGQGLEWIGGINPTNGNTDFNE | SEQ ID NO: |
| variable region | NFKNKATLTVDKSSSTVYMRLSGLTSEDSAVYYCSRSKYGYAWFAYWGRGTLVTVSAAKTTP | 13 |
| sequence of | PSVY | |
| 2166G4 clone | | |
| antibody | | |
| (Protein) | | |
| Heavy chain | GAGGTCCAGCTGGAGGAGTCTGGGGCTGAACTGGTGAAGCCTGGGGCTTCAGTGAAGTTGTC | SEQ ID NO: |
| variable region | CTGCAAGGCTTCTGGCTACTCCTTCACCAGCTACTATATGTACTGGGTGAAGGAGAGGCCTG | 14 |
| sequence of | GACAAGGCCTTGAGTGGATTGGGGGGATCAATCCTACCAATGGTAATACTGACTTCAATGAG | |
| 2166G4 clone | AACTTCAAGAACAAGGCCACACTGACTGTAGACAAATCCTCCAGCACAGTCTATATGCGACT | |
| antibody (DNA) | CAGCGGCCTGACATCTGAGGACTCTGCGGTCTATTATTGTTCAAGATCTAAATATGGTTACG | |
| | CCTGGTTTGCTTACTGGGGCCGAGGGACTCTGGTCACTGTCTCTGCAGCCAAAACGACACCC | |
| | CCATCTGTCTAT | |
| Light chain | DIVLTQSHASLTVSLGQRAAISCRSSQSVNTSAYSYVHSYQQRPGQPPKLLIKYASNLESGV | SEQ ID NO: |
| variable region | PARFSVSGSGTDFTLNIHSVEEEDPTTYDCQHSWEIHSRSARGQSWKYNGLMLQTVS | 15 |
| sequence of | | |
| 2166G4 clone | | |
| antibody | | |
| (Protein) | | |
| Light chain | GACATTGTGCTGACCCAATCTCACGCTTCCTTAACTGTATCTCTGGGGCAGAGGGCCGCCAT | SEQ ID NO: |
| variable region | CTCATGCAGGTCCAGCCAAAGTGTCAATACATCTGCCTATAGTTATGTGCACTCCTACCAAC | 16 |
| sequence of | AGAGACCAGGCCAGCCACCCAAACTCCTCATCAAGTATGCATCCAACCTAGAATCTGGGGTC | |
| 2166G4 clone | CCTGCCAGGTTCAGTGTCAGTGGGTCTGGGACAGACTTCACCCTCAACATCCATTCTGTGGA | |
| antibody (DNA) | GGAGGAGGATCCTACAACATATGACTGTCAGCACAGTTGGGAGATTCATTCACGTTCGGCTC | |
| | GGGGGCAAAGTTGGAAATATAACGGGCTGATGCTGCAAACTGTATCC | |
| NG2/CSPG4 | MQSGPRPPLPAPGLALALTLTMLARLASAASFFGENHLEVPVAT | SEQ ID NO: |
| ectodomain | ALTDIDLQLQFSTSQPEALLLLAAGPADHLLLQLYSGRLQVRLVLGQEELRLQTPAET | 17 |
| | LLSDSIPHTVVLTVVEGWATLSVDGFLNASSAVPGAPLEVPYGLFVGGTGTLGLPYLR | |
| | GTSRPLRGCLHAATLNGRSLLRPLTPDVHEGCAEEFSASDDVALGFSGPHSLAAFPAW | |
| | GTQDEGTLEFTLTTQSRQAPLAFQAGGRRGDFIYVDIFEGHLRAVVEKGQGTVLLHNS | |
| | VPVADGQPHEVSVHINAHRLEISVDQYPTHTSNRGVLSYLEPRGSLLLGGLDAEASRH | |
| | LQEHRLGLTPEATNASLLGCMEDLSVNGQRRGLREALLTRNMAAGCRLEEEEYEDDAY | |
| | GHYEAFSTLAPEAWPAMELPEPCVPEPGLPPVFANFTQLLTISPLVVAEGGTAWLEWR | |
| | HVQPTLDLMEAELRKSQVLFSVTRGARHGELELDIPGAQARKMFTLLDVVNRKARFIH | |
| | DGSEDTSDQLVLEVSVTARVPMPSCLRRGQTYLLPIQVNPVNDPPHIIFPHGSLMVIL | |
| | EHTQKPLGPEVFQAYDPDSACEGLTFQVLGTSSGLPVERRDQPGEPATEFSCRELEAG | |
| | SLVYVHRGGPAQDLTFRVSDGLQASPPATLKVVAIRPAIQIHRSTGLRLAQGSAMPIL | |
| | PANLSVETNAVGQDVSVLFRVTGALQFGELQKQGAGGVEGAEWWATQAFHQRDVEQGR | |
| | VRYLSTDPQHHAYDTVENLALEVQVGQEILSNLSFPVTIQRATVWMLRLEPLHTQNTQ | |
| | QETLTTAHLEATLEEAGPSPPTFHYEVVQAPRKGNLQLQGTRLSDGQGFTQDDIQAGR | |
| | VTYGATARASEAVEDTFRFRVTAPPYFSPLYTFPIHIGGDPDAPVLTNVLLVVPEGGE | |
| | GVLSADHLFVKSLNSASYLYEVMERPRHGRLAWRGTQDKTTMVTSFTNEDLLRGRLVY | |
| | QHDDSETTEDDIPFVATRQGESSGDMAWEEVRGVFRVAIQPVNDHAPVQTISRIFHVA | |
| | RGGRRLLTTDDVAFSDADSGFADAQLVLTRKDLLFGSIVAVDEPTRPIYRFTQEDLRK | |
| | RRVLFVHSGADRGWIQLQVSDGQHQATALLEVQASEPYLRVANGSSLVVPQGGQGTID | |
| | TAVLHLDTNLDIRSGDEVHYHVTAGPRWGQLVRAGQPATAFSQQDLLDGAVLYSHNGS | |
| | LSPRDTMAFSVEAGPVHTDATLQVTIALEGPLAPLKLVRHKKIYVFQGEAAEIRRDQL | |
| | EAAQEAVPPADIVFSVKSPPSAGYLVMVSRGALADEPPSLDPVQSFSQEAVDTGRVLY | |
| | LHSRPEAWSDAFSLDVASGLGAPLEGVLVELEVLPAAIPLEAQNFSVPEGGSLTLAPP | |
| | LLRVSGPYFPTLLGLSLQVLEPPQHGALQKEDGPQARTLSAFSWRMVEEQLIRYVHDG | |
| | SETLTDSFVLMANASEMDRQSHPVAFTVTVLPVNDQPPILTTNTGLQMWEGATAPIPA | |
| | EALRSTDGDSGSEDLVYTIEQPSNGRVVLRGAPGTEVRSFTQAQLDGGLVLFSHRGTL | |
| | DGGFRFRLSDGEHTSPGHFFRVTAQKQVLLSLKGSQTLTVCPGSVQPLSSQTLRASSS | |
| | AGTDPQLLLYRVVRGPQLGRLFHAQQDSTGEALVNFTQAEVYAGNILYEHEMPPEPFW | |
| | EAHDTLELQLSSPPARDVAATLAVAVSFEAACPQRPSHLWKNKGLWVPEGQRARITVA | |
| | ALDASNLLASVPSPQRSEHDVLFQVTQFPSRGQLLVSEEPLHAGQPHFLQSQLAAGQL | |
| | VYAHGGGGTQQDGFHFRAHLQGPAGASVAGPQTSEAFAITVRDVNERPPQPQASVPLR | |
| | LTRGSRAPISRAQLSVVDPDSAPGEIEYEVQRAPHNGFLSLVGGGLGPVTRFTQADVD | |
| | SGRLAFVANGSSVAGIFQLSMSDGASPPLPMSLAVDILPSAIEVQLRAPLEVPQALGR | |
| | SSLSQQQLRVVSDREEPEAAYRLIQGPQYGHLLVGGRPTSAFSQFQIDQGEVVFAFTN | |
| | FSSSHDHFRVLALARGVNASAVVNVTVRALLHVWAGGPWPQGATLRLDPTVLDAGELA | |
| | NRTGSVPRFRLLEGPRHGRVVRVPRARTEPGGSQLVEQFTQQDLEDGRLGLEVGRPEG | |
| | RAPGPAGDSLTLELWAQGVPPAVASLDFATEPYNAARPYSVALLSVPEAARTEAGKPE | |
| | SSTPTGEPGPMASSPEPAVAKGGFLSFLEANMFSVIIPMCLVLLLLALILPLLFYLRK | |
| | RNKTGKHDVQVLTAKPRNGLAGDTETFRKVEPGQAIPLTAVPGQGPPPGGQPDPELLQ | |
| | FCRTPNPALKNGQYWV | |

According to a preferred embodiment the antibody is an antibody fragment, more preferably selected from the group consisting of: half-IgG, Fc, VH, VHH, VL, VLL, F(ab′)2, Fab, Fab′ and Fv.

F(ab′)2, Fab, Fab′ and Fv are antigen-binding fragments that can be generated from the variable region of IgG and IgM. These antigen-binding fragments vary in size (MW), valency and Fc content.

Fc fragments are generated entirely from the heavy chain constant region of an immunoglobulin.

According to a further preferred embodiment, the antibody is diabody or a scFv obtained with the method generally used for these purposes.

A further aspect of the present invention refers to a pharmacological composition comprising a therapeutic amount of the antibody, in its entirety or single chain variable fragment, or scFv of the invention that is active against NG2/CSPG4, or any other portion of the antibody, for example, embodied in a bispecific antibody construct (e.g. an antibody binding both NG2/CSPG4 and another cell surface molecule and/or antigen) or a fusion protein comprised of a therapeutic amount of the antibody, in its entirety or single chain variable fragment, or scFv, an another therapeutic molecule, such as a cytochine or an aptoptosis-inducing agent (e.g TRAIL or Fas Ligand), or a CAR T (Chimeric Antigen Receptor T cell) construct in which the scFv derived from the portion of the antibody of the invention is expressed on the surface of a CAR T cell, such as to simultaneously engaging NG2/CSPG4 expressing cells and endogenous T cells and favor the maintenance of the CAR T cell in proximity to the cancer cell to trigger the activation of the engaged T cell. The invention further relates to an expression vector comprising the nucleotide sequences here disclosed. Said expression vector comprises all the nucleotide sequences requested for the antibody expression in a host cell.

Therefore, a further aspect of the present invention is a host cell comprising said expression vector.

The sequences necessary for expression in prokaryotes regard for example a promoter and, optionally, an operator sequence, a ribosome-binding site and possibly other 3 0 sequences. For eukaryotic cell expression, sequences such as promoters, enhancers, termination signals and polyadenilation are required.

Said expression vector may also comprises signal sequences for targeting the antibody or its variant disclosed above in a particular cellular compartment.

Said vector may further comprise any selection gene whose expression is easily used for selecting the recombinant host cells transformed with the vector.

A further aspect of the present invention refers to a composition comprising the antibody disclosed above and pharmaceutically acceptable excipient generally used for this purpose.

The antibody, or the antibody fragment, or the diabody, or the scFv, or the composition of the invention is preferably used for treating a disease whose treatment relies upon external induction of apoptosis and/or autophagy in the cells responsible for the disease. In other words, the invention contemplates any disease for which programmed cell death, such as apoptosis and/or autophagy, induced by external means can effectively be exploited for curative purposes.

Therefore, a further aspect of the invention refers to a method for treating a disease dependent upon resistance to programmed cell death. It involves a step of administration to an individual, suspected to be or affected by such disease, of an effective amount of the antibody or the composition of the invention. The administration to the individual allows the binding of the administered antibody to the NG2/CSPG4 ectodomain expressed on the membrane of the cells responsible, or associated with the disease to be treated.

Preferably, the antibody or the composition is administered systemically or locally, more preferably by injection, or by any other means allowing the antibody to most effectively reach the target cells.

For the scope of the present invention, the disease of interest is preferably cancer, primarily solid, hematological malignancies or any other diseases requiring a curative elimination of cells expressing NG2/CSPG4 are also contemplated.

More preferably, but not exclusively, said disease is cancer, preferably a cancer selected from the group consisting of: any variant, subtype or histotype of melanomas, soft-tissue, cartilage or bone sarcomas, head or neck carcinomas, colorectal carcinomas, lung carcinomas, prostate carcinomas, breast carcinomas, skin carcinomas, mesotheliomas, hematological neoplasia or Central Nervous System (CNS) and Peripheral Nervous System (PNS) tumors.

The antibody, or the antibody fragment, or the diabody, or the scFv, or the composition is preferably used for treating and/or preventing formation of secondary metastatic and non-metastatic lesions, and relapsing local, loco-regional and distant tumor formations.

The antibody or the antibody fragment, or the diabody, or the scFv of the invention functions by recognizing and binding with high-affinity the NG2/CSPG4 transmembrane PG, preferably discrete NG2/CSPG4 isoformes, expressed by cells and by activating in these cells defined programmed cell death pathways. These pathways drive the cell to its death by apoptotic, preferable caspase-dependent, and autophagic mechanisms, or related mechanisms of programmed cell death, in a manner apparently independent of other intervening extracellular factors.

According to a preferred embodiment, the antibody, or the antibody fragment, or the diabody, or the scFv, or the composition of the invention is administered (used) in combination with other drugs, preferably with other conventional or experimental targeted and non-targeted chemotherapeutic agents. Alternatively, the antibody, or the antibody fragment, or the diabody, or the scFv, or the composition of the invention stands alongside other therapeutic treatments, such as surgical resection and/or radiotherapy.

Being NG2/CSPG4 expression on cancer cells putatively associated with cancer insurgence, progression and recurrence, a further aspect of the present invention is the use of the antibody of the invention for diagnostic use, preferably as a biomarker for the routine clinical monitoring of cancer patients and/or for in vivo whole-body or local diagnostic imaging procedure and/or for identification, enumeration and isolation of cancer circulating cells.

A further aspect of the present invention refers to a naked polypeptide formulation of the naturally produced and/or humanized and/or genetically engineered and/or recombinantly produced and/or synthetically derived compound active in inducing cell death and including at least one of the anti-NG2/CSPG4 antibody, or fragments thereof, of the invention.

### EXAMPLE

In the present invention, four murine monoclonal antibodies were produced by using a recombinant, eukaryotic fragment corresponding to the extracellular portion of the NG2/CSPG4 transmembrane proteoglycan and by adopting the conventional Koehler and Milstein immunization and hybridoma production method (Harlow E. and Lane D., “Antibodies: a laboratory manual”, Cold Spring Harbor Laboratory, 1988; Howard G. C. and Kaser M. R., “Making and using antibodies: a practical handbook”, CRC Press Taylor & Francis Group, 2006)

Isotyping of the NG2/CSPG4 monoclonal antibodies

In order to identify classes, subclasses, and type of light chains that belong to immunoglobulins produced by hybridomas, Pierce® Rapid ELISA Mouse antibody Isotyping Kit was used. This assay uses ELISA strip-well plates with individual wells, pre-coated with either anti-mouse heavy-chain capture antibody (anti-IgG1, IgG2a, IgG2b, IgG3, IgA and IgM), or anti-mouse light-chain capture antibody (kappa or lambda). This approach eliminates the need to purify and immobilize an antigen for isotyping.

Supernatants of hybridoma cells grown in Ig-depleted serum were diluted 1:10 in TBS (Tris-buffered saline) and dispensed into the plates. Antibody binding was detected using polyvalent secondary HRP-conjugated polyclonal antibodies, and the plates were incubated at room temperature under shaking for 1 hour in the dark. Subsequently, wells were thoroughly rinsed with wash buffer, and then incubated with 3,3′,5,5′-Tetramethylbenzidine (TMB) Liquid substrate (Sigma-Aldrich) for 10 min and absorbance of the colorimetric reaction was measured at 450 nm using a microplate adsorbance reader.

All antibodies were found of the IgG1 class.

Antibody binding to putative NG2/CSPG4 isoforms as evidenced by cell-ELISA and flow cytometry

In order to evaluate the ability of the antibodies to recognize diverse NG2/CSPG4 isoforms they were initially assayed on a panel of NG2/CSPG4-expressing cells by using a cell-ELISA approach on live and fixed cells, followed up by conventional flow cytometry. The cellular models used for these experiments were the following cell lines: SK-LMS1 and SK-UT-1 (human leiomyosarcoma cell lines), HT1080^{NG2+}(human fibrosarcoma cell line immunosorted by FACS using the commercially available pan-anti-NG2/CSPG4 antibody 9.2.27), 143B (human bone osteosarcoma), A375 (human melanoma) and M2 (human melanoma). For cell-ELISA on fixed cells, test cells were seeded in 96 multiwell plates in complete growth medium (10,000 cells/well). After fixation with 2% PFA for 30 min, endogenous peroxidase activity was neutralized by incubation with 3% H₂O₂ for 15 min at room temperature. Blocking of non-specific binding sites was performed by further incubation of the cells with blocking buffer (PBS with 2% BSA; 10% sucrose; 0,1% NaN₃) for 30 min at room temperature. Cells were finally incubated overnight at 4° C. with the anti-NG2/CSPG4 antibodies, diluted as follows: supernatants, 1:1-1:2 and ascites fluids, 1:50 in blocking buffer. The Streptavidin/Biotin (Thermo Scientific TS-125-HR and TM-125-BN) and TMB (T4444 Sigma) detection method was used for the detection of the binding of the antibodies to the antigen, according to the procedure recommended by the manufacturer. All assays were performed in triplicate.

For cell-ELISA on live cells, test cells were seeded in 96 multiwell plates in complete growth medium (melanoma cells at 25,000 cells/well; sarcoma cells at 20,000 cells/well). Blocking of non-specific binding sites on cells was performed by incubating them with sterile blocking buffer (PBS and 2% BSA; 10% sucrose) for 30 min at 37° C. Then, cells were further incubated for 2 hours at 37° C. with the anti-NG2/CSPG4 antibodies, diluted as follows: supernatants, 1:1-1:5 and ascites fluids, 1:50-1:100 in blocking buffer. After fixation with 2% PFA for 30 min, endogenous peroxidase activity was quenched by incubation with 3% H₂O₂ for 15 min at room temperature. Detection of bound antibody was similarly detected through the Streptavidin/Biotin (Thermo Scientific TS-125-HR and TM-125-BN) and TMB (T4444 Sigma) system described above. All assays were performed in triplicate. The outcome of these experiments is summarized in Table 2. Antibodies derived from the same immunization and spleen fusion protocol, but found to be did not react with certain cell lines, these cases were adopted as internal negative controls.

TABLE 2

| | | | | | cell-ELISA (live cells) | | | |
|---|---|---|---|---|---|---|---|---|
| | cell-ELISA (fixed cells) | | | | | | SK- | |
| CLONE | A375 | SK-UT1 | 143B | M2 | A375 | SK-UT1 | LMS1 | M2 |
| 2161D2/C2 | 11 | 1 | 9 | 0 | 42 | 15 | 25 | 0 |
| 2164H5/E11 | 0 | 61 | 21 | 0 | 5 | 20 | 0 | 0 |
| 2166G4/C10/D1 | 40 | 37 | 73 | 0 | 35 | 17 | 38 | 0 |
| 2172B12 | 4 | 15 | 0 | 0 | 31 | 57 | 5 | 0 |

Each antibody was found to exhibit a different reactivity pattern, supporting the idea that it recognizes a distinct NG2/CSPG4 isoform. In addition, to note was that antibodies display different binding patterns when tested on live or fixed cells, which further indicates that they possess diverse affinities for native and partly or entirely denatured forms of NG2/CSPG4.

To then confirm by a different method the relative expression of NG2/CSPG4 isoforms detected by the antibodies on tumor cells, flow cytometric analyses were performed using the commercially available pan-human NG2/CSPG4 monoclonal antibody 7.1 PE-labeled (Beckman-Coulter) as reference. While untreated cells and cells treated with non-specific immunoglobulines were used as a negative control.

Cells were detached with Accutase or EDTA, collected in tubes and counted. A minimal amount of 300,000 cells for was used for each antibody or control sample. Cells were rinsed with PBS (with 1% FBS) by centrifugation at 0.3 RCF, then they were incubated with antibodies in the dark at 4 ° C. for 30 minutes. After antibody incubation, cells were rinsed twice with PBS by centrifugation at 0.3 RCF, and resuspended in PBS (with 1% FBS). All samples were analyzed using a FACScan flow cytometer (BD Biosciences) and the resulting bindings analysed using the flow cytometer software BD Cell Quest Pro™. Relative NG2/CSPG4 expression on the different tumor cell lines is summarized in Table 3.

TABLE 3

| | NG2/CSPG4 expression (%) | | | | | | |
|---|---|---|---|---|---|---|---|
| CLONE | SK-LMS1 | SK-UT1 | COLO38 | HT1080^{NG2+} | A375 | M2 | 143B |
| 2161D2/C2 | 3 | 12 | 19 | 0 | 74 | 0 | 86 |
| 2164H5/E11 | 9 | 1 | 11 | 92 | 89 | 0 | 57 |
| 2166G4/C10/D1 | 3 | 3 | 48 | 26 | 71 | 0 | 0 |
| 2172B12/C10 | 64 | 47 | 16 | 94 | 91 | 0 | 32 |
| 7.1-PE (control) | 89 | 64 | 94 | 38 | 92 | 96 | 62 |

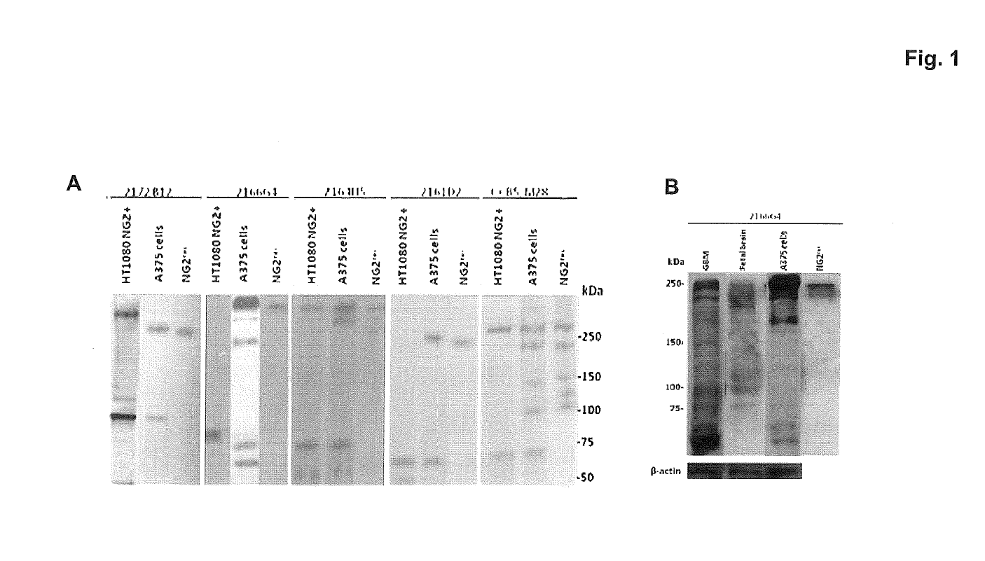

The outcome of these experiments further corroborated a putative expression of different antibody-reactive isoforms on tumor cells and specifically confirmed that M2 melanoma cells express a NG2/CSPG4 isoform(s) not recognized by these reagents. Differential expression of NG2/CSPG4 isoforms on the model cell lines, and the extracellular proteolytic processing of these isoforms, was eventually also investigated by immunoblotting using lysates of A375 and HT1080^{NG2}+cells (Fig.1A). For this purpose, cells were directly lysed in sample buffer and the lysates resolved by SDS-PAGE under reducing conditions on 5% or 4-15% gradient gels, followed by transferring onto nitrocellulose membranes and incubation with the indicated anti-NG2/CSPG4 antibodys. Recombinant NG2/CSPG4 (NG2^{rec}) was used as reference. In these experiments, NG2/CSPG4 isoforms and their proteolytic fragments were also studied on lysates of pericyte sprouts of fetal brain neovessels and in glioblastoma multiforme lesions (GMB; Fig.1 B). Sections from brain areas taken adjacently to sections with abundant angionenesis and enrichment of NG2/CSPG4-expressing pericytes. Immunoblotting of 8-actin with a commercial polyclonal antibody was used for normalization of the gel lane loading.

The banding pattern observed for A375 cells indicated that these cells express mainly two NG2/CSPG4 isoforms: one an apparent MW greater than 250 kDa and the other of a smaller size. These isoforms were primarily recognized by antibodies 2166G4/C10/D1 and 2164H5/E11. Conversely, antibodies 2172B12/C10 and 2161 D2/C2 only recognized the smaller isoform expressed by A375 cells. HT1080^{NG2+} cells did not seem to express the smaller isoform, but displayed the larger isoform recognized by antibodies 2172B12/C10 and 2164H5/E11. Antibody 2161D2/C2 was found to recognize preferentially a proteolytically processed form(s) of the protein running between 100-120 kDa, while antibody 2172B12/C10 showed a broader isoform reactivity documented by binding to different isoforms and their proteolytic fragments which had apparent MW is in the range of 75 to 250 kDa. Fetal brain NG2/CSPG4 isoforms were poorly recognized and it seemed that antibody 2166G4/C10/D1 was the one that in this tissue recognized the primary isoforms and their proteolytic fragments (FIG. 16).

These immunochemical analyses corroborate the distinct molecular identity of the NG2/CSPG4 isoforms recognized by the four antibodies and further indicate that some of them may specifically detect naturally occurring proteolytic forms of the proteoglycan generated at the time or following cell surface shedding of its extracellular portion. Subcellular localization of NG2/CSPG4 isoforms detected by the antibodies as 2 0 determined by in vitro immunolabeling

Tumor cell lines A375, Hs578T (human breast carcinoma) and HT1080^{NG2+}and primary human brain vascular pericytes (HBVP) were grown to subconfluence and stained with the different antibodies. For this purpose, 25,000 cells were seeded into each well of a 24-well plate, in which a round glass coverslip had been placed at the bottom. Tumor cell lines were routinely grown in DMEM medium supplemented with 10% FBS, whereas HBVP were cultured in PM medium supplemented with 1% penicillin/streptomycin, 1% PGS and 2% FBS as recommended by the supplier (Lonza). After 24 hours of culture, cells were fixed at room temperature with 4% PFA for 20 min and non-specific binding sites were blocked by incubation with 20% Normal Goat Serum (NGS) in PBS for 30 min at room temperature. Cells were then incubated overnight at 4° C. with antibody supernatants diluted 1:1-1:2 in NGS, or purified antibodies used at 150-300 ng/μ1. After washes, coverslips were incubated for 1 hour at room temperature with a goat anti-mouse secondary antibody conjugated to the Alexa Fluor® 594 dye diluted 1:250 in PBS. Next, they were washed and incubated for 5 minutes with Hoechst (Sigma Aldrich) diluted 1:1,000 in PBS. Coverslips were finally washed and mounted on microscope slides with Non-Fluorescing Mounting Aqua-Poly/Mount (Polisciences, Inc) and viewed under a fluorescence microscope using appropriate a filter sets.

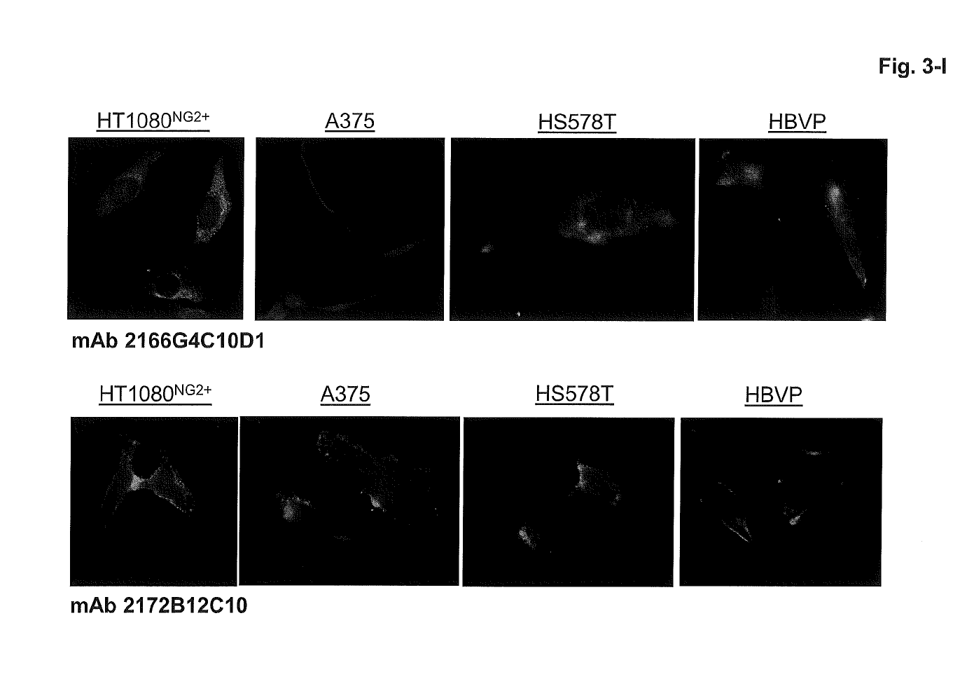

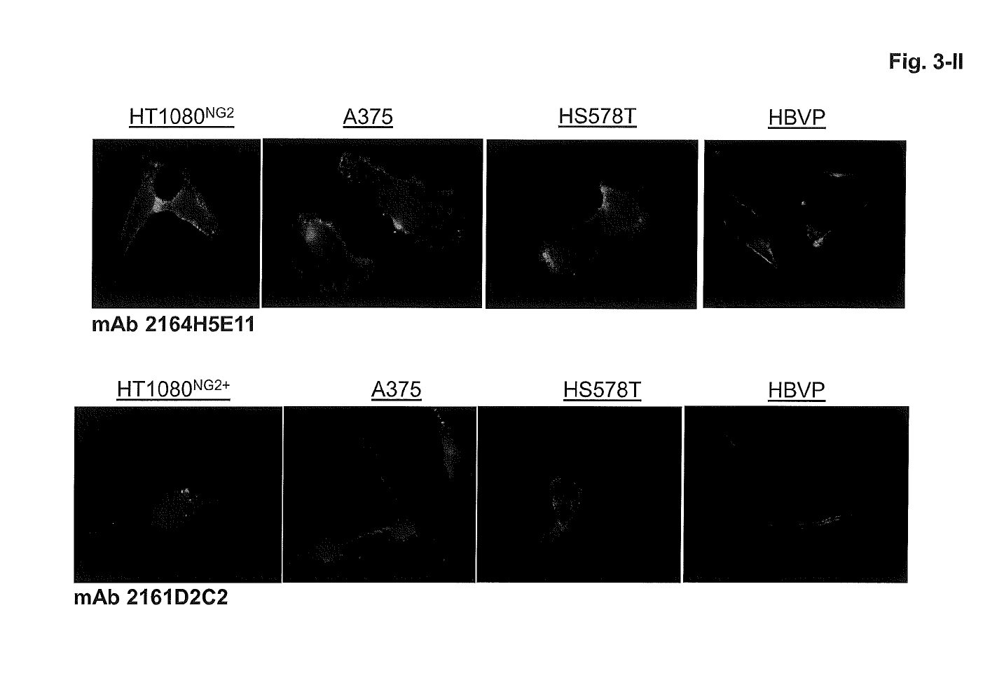

As expected, NG2/CSPG4 was primarily immunolocalized on the cell membrane of both tumor cell lines, while the different staining intensity is believed to reflect the efficiency with which each antibody detected the corresponding isoform in that specific cellular compartment. In some cases, staining for NG2/CSPG4 was also observed in intracellular locations, suggesting that some of the antibodies recognized the nascent NG2/CSPG4 polypeptide and may additionally have identified spontaneously internalized NG2/CSPG4 molecules (FIG. 3I-II).

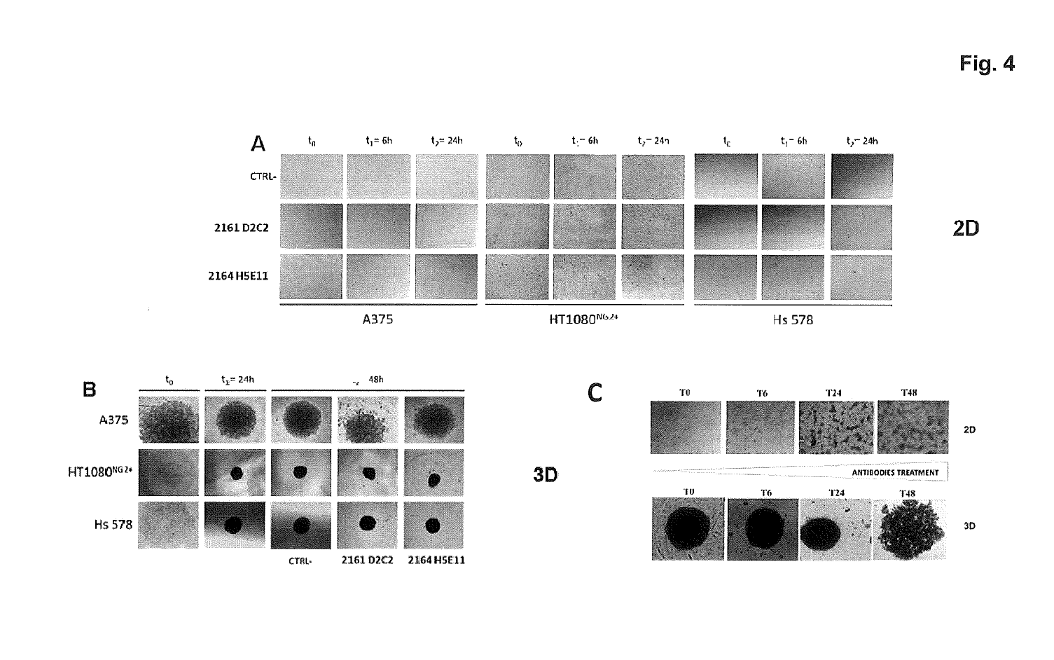

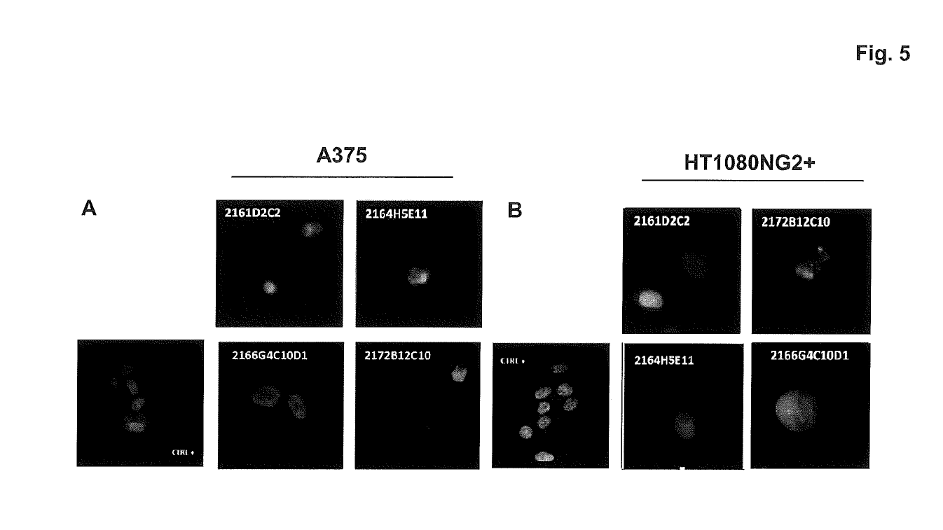

Effects of anti-NG2/CSPG4 antibodies on cell-substrate adhesion and migration in vitro. To evaluate the ability of the antibodies to neutralize the function of NG2/CSPG4 in the control of cellular interactions, with particular reference to the role exerted by the proteoglycan in the regulation of cell-substrate adhesion and cell motility, the above cell lines were incubated with the antibodies and monitored by microscopy in 2D monolayer culture of as 3D tumor spheroids generated by prior culture of the cells on Poly-HEMA substrates. Cell motility studies were carried out adopting the conventional scratch-based wound-healing type assay as a paradigm. In all these experiments and the assays described below, we routinely used anti-NG2/CSPG4 antibodies derived from the same immunization and spleen fusion protocol and possessing either a marked ability to react with the native antigen expressed on the cell surface or lacking such ability, but which had not been found to affect cell behavior in pilot tests. For these experiments the above described melanoma, fibrosarcoma and breast carcinoma cell lines were seeded in 24 well plates and examined at 6, 24, 48 hours time intervals, with and without addition of purified anti-NG2/CSPG4 antibodies administered at 20-30 ng/ml in serum-free growth medium. After microscopic end-point evaluation, cells were lysed and processed for immunoblotting with antibodies against cytoskeletal components and antibodies to cell death markers (see below). Treatment of cells with antibodys 2161 D2/C2 and 2164H5/E11 caused cell shape changes and detachment of the cells within 24 hours both in 2D (FIG. 4A-5A) and in spheroids 3D settings (FIG. 4B-5B).

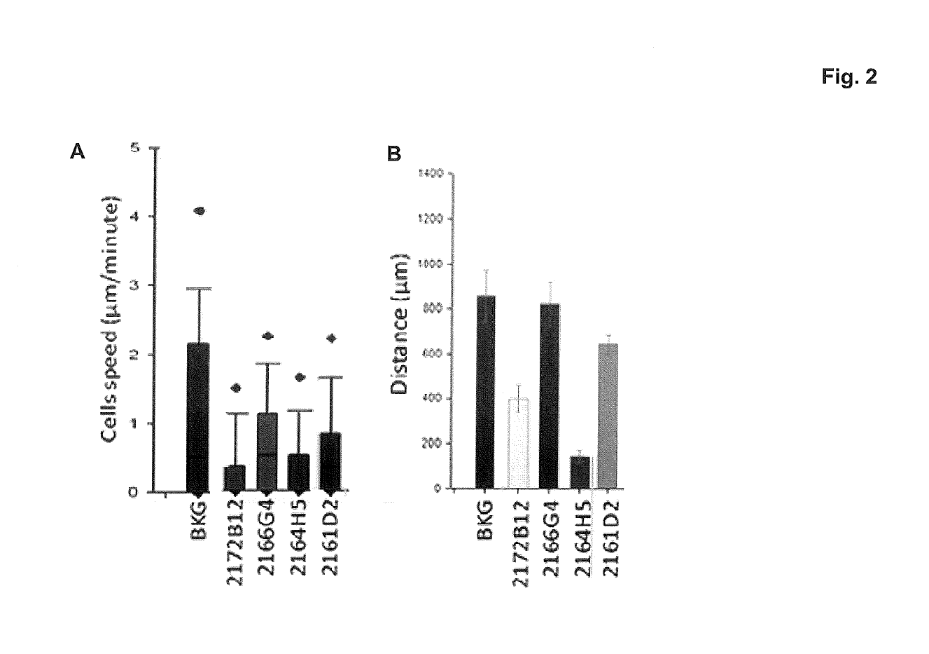

For cell migration assays, HT1080^{NG2+} cells were selected as a preferential model because of their pronounced locomotory abilities. Confluent cells were starved and then scratched in the middle of the monolayer and exposed to diverse quantities of the purified anti-NG2/CSPG4 antibodies as described above. Cell migration was monitored by phase-contrast video time-lapse microscopy for 24 hours. Cell migration rate was assessed by mean displacement (MD, i.e. the mean distance (mm) traveled per minute) of the cell centroid. The migratory potential of cell population was then be expressed as the average mean displacement (AMD) of all cells in the analysis. In order to determine how the manual selection of the centroid positions of cells influences the calculated migration rates of individual cells and cell populations, cells were manually marked by using a point-and-click system. In this manual cell tracking method, a subset of cells from each time-lapse video was selected for analysis. This subset is assumed to represent the whole cell population. The results are summarized in FIG. 2. In the first histogram (FIG. 2A) cells speed for each different well is plotted; in the second histogram (FIG. 2B) the covered distances is plotted for each antibody. The results show that anti-NG2/CSPG4 antibodies are able to differentially interfere with cell motility, an effect that was particularly evident for antibodies 2164H5/E11 and 2172B12/C10. Incubation of NG2/CSPG4 expressing cells with the antibodies induces programmed cell death.

The observed changes in cell shape observed after incubation of cells with the anti-NG2/CSPG4 suggested that engagement of the proteoglycan with these specific antibodies could uniquely induce intracellular alterations triggering programmed cell death. To verify this possibility A375 and HT1080^{NG2+} cells were time- and dose-dependently treated with the four antibodies of this invention and examined through a proprietary multiparametric cell death detection platform. Staurosporine was used at a concentration of 500 nM as positive control. Preliminary screening experiments established that purified antibodies were most effective at a concentration of 20-30 ng/μl and were therefore consistently used at this concentration. To effectively detect apoptotic cells, we initially used the DeadEnd™ Fluorometric TUNEL Assay combined with PI staining. For this purpose, cells cultivated on coverslips within 24 well plates were fixed with 4% PFA for 20 min at room temperature, followed by extensive washing with PBS and permeabilization with 0.1% Triton X-100 in PBS for 10 minutes. Equilibration Buffer was then added at room temperature for 5-10 minutes was added in each well and cells were further incubated with Equilibration Buffer supplemented with Nucleotide Mix and rTdT Enzyme at 37° C. for 60 min inside a humidified chamber to allow the tailing reaction to occur. Incubation buffer was then removed and 2X SSC (Saline Sodium Citrate) was added for 15 min at room temperature. The samples were finally extensively with PBS to remove unincorporated fluorescein-12-dUTP and incubated with PI (1μg/ml in PBS) for 15 minutes at room temperature in the dark. Coverslips were removed from the wells washed with deionized water for 5 minutes at room temperature, mounted on microscope slides and viewed under a fluorescence and confocal laser microscopy using the appropriate filter sets.

The results reported in FIG. 5 document the differentially ability of the antibodies to induce programmed cell death in the two cell lines.

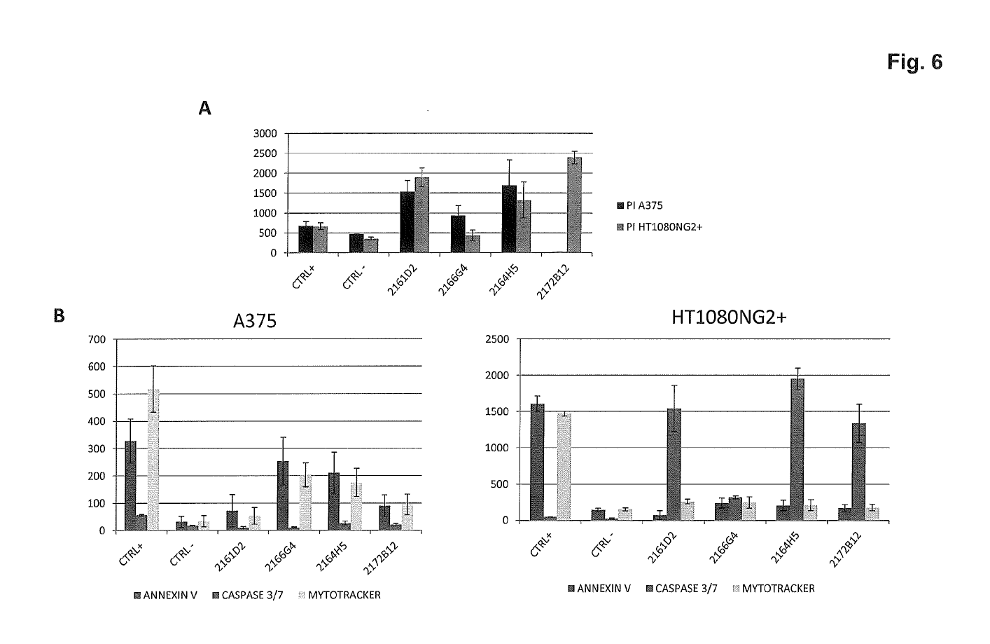

These experiments were also repeated using the Acumen eX3 microplate laser cytometer (TTP Labtech Ltd, UK). When comparing to the control, the NG2/CSPG4-specific antibodys 2161 D2C2 and 2164H5E11 increased by a three-fold the number of apoptotic A375 and HT1080^{NG2+}cells; whereas antibody 2166G4/C10/D1 had no effect on HT1080^{NG2+}, but doubled the A375 apoptotic cells number. Antibody 2172B12/C10 promoted full apoptosis in HT1080^{NG2+}, but not A375 cells (FIG. 6).

To further examine the extent to which the different antibodies were able to promote the different phases of apoptosis we applied the multiparametric assay additionally involving detection of phosphatidylserine translocation through APC-tagged Annexin V and mitochondrial membrane potential changes assessed through the Mytotraker Red marker (Life Technologies, Inc.). In the same multiparametric assay, the activation of Caspase 3/7 was also investigated by CellEventTMCaspase-3/7 Green Detection Reagent (FIG. 6).

We observed that the NG2/CSPG4-specific antibodys 2161 D2/C2 had a weak effect, whereas antibodys 2166G4/C10/D1 and 2164H5/E11 doubled the apoptotic A375 cells compared to the control. All antibodys were effective on HT1080^{NG2+} cells, in particular when evaluating caspase activation, although 2166G4/C10/D1 showed the weakest action.

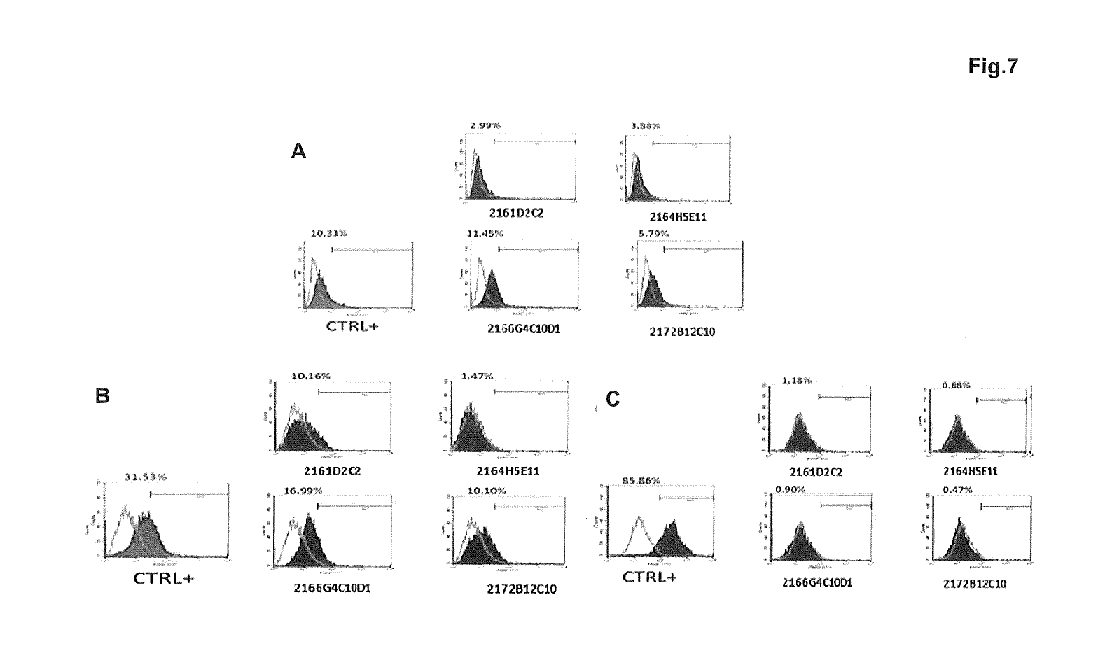

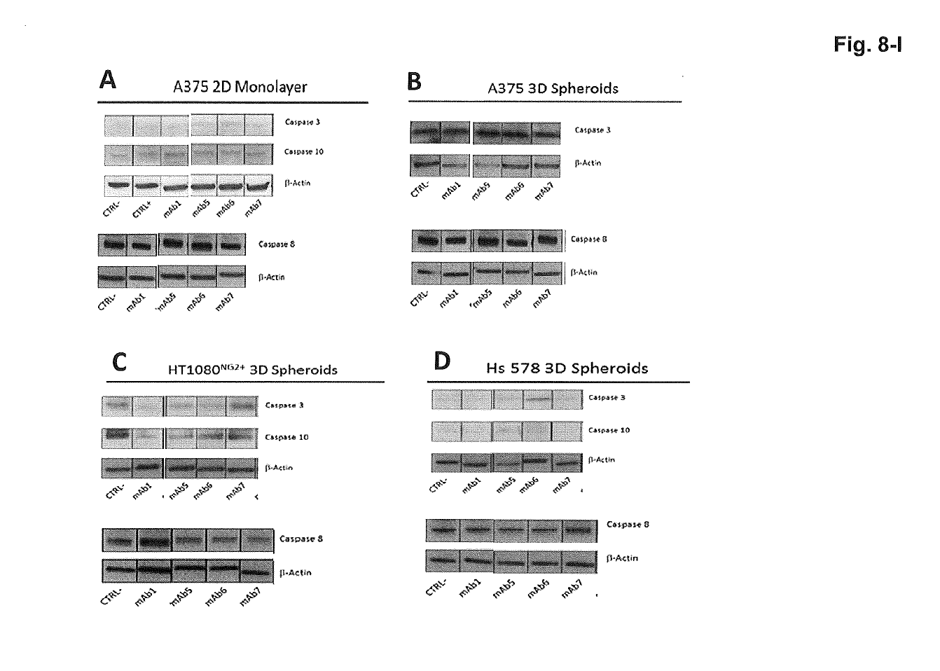

We next added another apoptotic marker to the system and assessed PARP cleavage by flow cytometry. To this end, A375 (FIG. 7A), HT1080^{NG2}+(FIG. 7B), and Hs578T (FIG. 7C) cells, cultured as monolayers, were treated with anti-NG2/CSPG4 antibodies as described above and examined by flow cytometry with an antibody specifically recognizing cleaved PARP. These experiments were complemented by Western blotting experiments using analogous antibodies directed against cleaved PARP. The results of these experiments indicate a stronger apoptosis induction in HT1080^{NG2+} and A375 cell lines after treatment with 2166G4/C10/D1 compared to that seen in Hs578T cells.

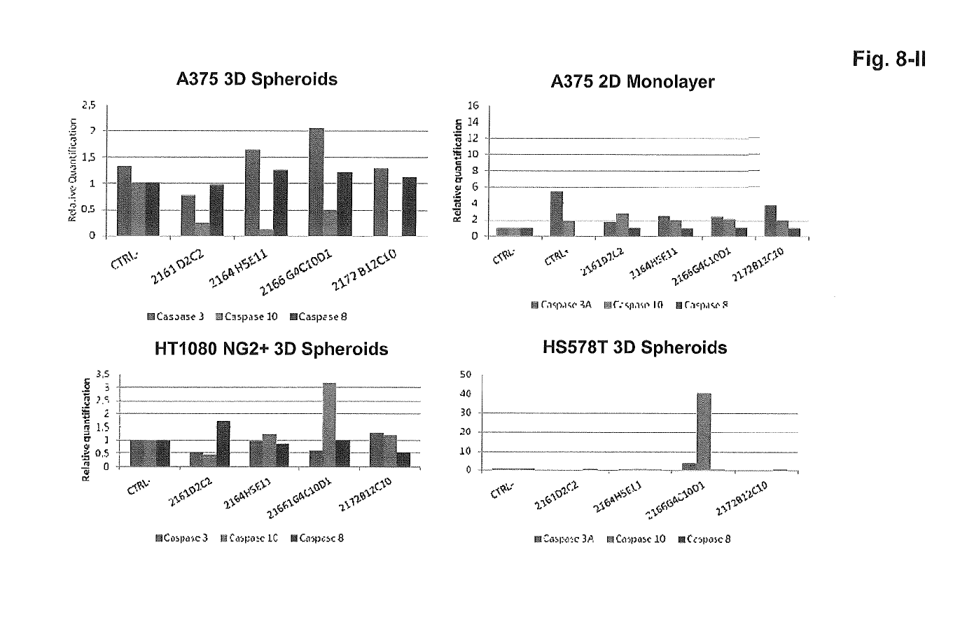

Corroborative, parallel detection of activated effector caspases was performed by immunoblotting on lysates from cells treated with antibodies in 2D monolayer and 3D multicellular spheroids settings. In these cases cell lysates were resolved by SDS-PAGE under reducing conditions, followed by transferring onto nitrocellulose membranes and immunoblotting with antibodies against pro-caspase forms and the activated forms of caspases 3, 8 and 10. Antibodies to I3-actin were used as internal normalization standard. Band intensities were analyzed through densitometry and normalized for the internal standard. Relative caspase activation was assessed by establishing the ratio between sample bands intensities and negative control. The results of these experiments are reported in FIG. 8 where it is evident that the anti-NG2/CSPG4 antibody displaying the greatest pro-apoptotic effect documented by caspase activation was antibody 2172B12/C10. In A375 cells cultured as 3D multicellular spheroids, all antibodies induced cleavage of both caspase 3 and 8 (FIG. 8 I-II). Conversely, caspase 10 seemed to be the primary caspase to be activated in HT1080^{NG2}+cells grown as 3D multicellular spheroids (FIG. 8 I-II), and the most effective antibody in this case was 2166G4/C10/D1. Caspase 8 activation was particularly prominent after treatment with antibody 2161 D2/C2, whereas caspase 3 activation was most strongly elicited by treatment with antibody 2172B12/C10.

Comprehensively, these sets of data highlight a differential ability of the different anti-NG2/CSPG4 antibodies to induce caspase-dependent apoptosis, which may tightly correlate with distinct isoform specificity of the antibodies and the documented differential expression of these putative isoforms on the different model cell lines. The findings further show that engagement of NG2/CSPG4 through the antibodies of this invention activates apoptotic events through the extrinsic pathway in the absence of canonical death receptor ligands.

Anti-NG2/CSPG4 antibodies induced programmed cell death through a dual pro-apoptotic and pro-autophagic action.

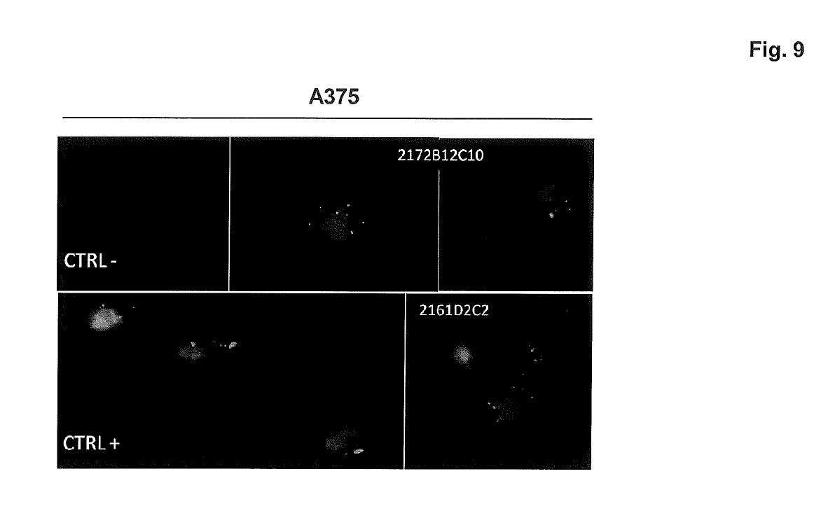

The possibility that the anti-NG2/CSPG4 antibodies of this invention could induce both caspase-dependent and autophagic programmed cell death was asserted by evaluating the expression of the two autophagic markers beclin-1 and LC3. To this end, A375 cells, were transiently transfected with a plasmid encoding the LC3-GFP fusion protein and treated with purified anti-NG2/CSPG4 antibodies as described above. As negative control we use untreated cells and as positive control cells treated with 100 nM Rapamicin for 24 and 48 hours. In untreated cells, LC3-GFP exhibited a diffuse cytoplasmic signal, whereas in antibody-treated cells undergoing autophagy, LC3-GFP chimeric proteins aggregated in autophagic vacuoles visualized as a punctuate cytoplasmic staining. Parallel autophagic evaluations were performed by flow cytometry using the Millipore FlowCellect FITC-LC3 Reporter Autophagy Assay kit. Although the signal appeared weak in all three cell lines (A375, FIG. 9A), HT1080NG2+(FIG. 9B) and Hs578T (FIG. 9C) cells, a significant peak shift asserted that authophagic phenomena took place in the cells following antibody treatment. Antibody 2172D6/B1 appeared the most effective anti-NG2/CSPG4 antibody in inducing autophagic cell death, which was most pronounced in the melanoma and breast carcinoma cell lines, whereas antibodies 2172B12/C10 and 2166G4/C10/D1 gave quantitatively distinct effects of the A375 and Hs578T cells. Even in this case, differential induction of autophagy by the different antibodies in the different cell lines lend support to a differential involvement of different NG2/CSPG4 isoforms in the programmed cell death induced by the different antibodies. Activation of autophagy in melanoma (A375) and soft-tissue sarcoma (HT1080NG2+) cell lines by anti-NG2/CSPG4 antibody treatment.

LC3 (microtubule-associated protein light chain 3), the most studied autophagy biomarker, was originally identified as a subunit of microtubule-associated proteins 1A and 1B (MAP1 LC3) and was later found to contain similarity to yeast protein Apg8/Aut7/Cvt5. Distributed ubiquitously in eukaryotes, LC3 is expressed as 3 splice variants/isoforms (LC3A, LC3B and LC3C) which undergo post-translational processing, wherein, the unprocessed form of LC3 is proteolytically cleaved by Atg4 protease to form LC3-I with carboxyterminal exposed glycine. During autophagy, this exposed glycine of LC3-I is conjugated by Atg7 (an E1-like activity), Atg3 (an E2-like conjugating activity) and by Atg12-Atg5-Atg16L multimers (E3-like ligase activity) to phosphatidylethanolamine (PE) moiety for generating LC3-II. The lipophilic character of PE group facilitates LC3-II insertion into autophagosomes membranes, and as a result LC3-II is degraded when autophagosomes fuse with lysosomes to form autolysosomes for lysus of intra-autophagosomal components by lysosomal hydrolases. Conversion of LC3I to LC3II when correlated with autophagosome numbers is considered as the best marker of autophagy because LC3-II is the only well-characterized protein which specifically localize to autophagic structures throughout autophagy (from phagophore to lysosomal degradation).

Levels of LC3-I and LC3-II were measured by quantitative Western Blot in melanoma and fibrosarcoma cell lines, using a specific primary antibody that detect both bands ˜17 and ˜19 kDa), corresponding respectively to LC3-II and LC3-I.

In particular, cells were treated with the 4 anti-NG2/CSPG4-specific mAbs and a negative control mAb, total protein content was extracted and resolved by SDS-PAGE under reducing conditions, followed by transfer onto nitrocellulose membranes and immunoblotting with mAbs able to detect LC3I-II. β-Actin has been used as internal standard.

Microtuble-associated protein light chain 3 (LC3) has been used as a specific marker to monitor autophagy. Upon induction of autophagy, LC3 is conjugated to phosphatidylethanolamine and targeted to autophagic membranes. Therefore, changes in LC3 localization have been used to measure autophagy. A transfection assay was conducted using a plasmid encoding the

Autophagosome marker LC3 fused with the fluorescent protein EGFP. When GFP-LC3 is expressed in cultured cells, punctate signals are simply observed by fluorescence microscopy.

A375 and HT1080NG2+ cells, were transiently transfected with a plasmid encoding the LC3-GFP fusion protein and treated with purified anti-NG2/CSPG4 antibodies. As negative control we use untreated cells and as positive control cells treated with 100 nM Rapamicin for 24 and 48 hours. In untreated cells, LC3-GFP exhibited a diffuse cytoplasmic signal, whereas in antibody-treated cells undergoing autophagy, LC3-GFP chimeric proteins aggregated in autophagic vacuoles visualized as a punctuate cytoplasmic staining.

The results reveal that all antibodies induce endogenous LC3 conversion in each cell lines.

Moreover, autophagy induction in A375 and HT1080 ^{NG2+}cells was assessed by estimating the percentage GFP-LC3 expressing cells. As result, an evident activation of autophagic pathway was found in cell lines treated with the anti-NG2/CSPG4 mAbs.

1. An antibody, recognizing and binding to an antigen comprising the ectodomain of the NG2/CSPG4 transmembrane proteoglycan, wherein said antibody induces cancer cell apoptosis and/or autophagy upon high-affinity binding to the antigen.

2. The antibody according to claim 1 characterized by:
SEQ ID NO: 18, 19 and SEQ ID NO: 20, 21 , 22, wherein SEQ ID NO: 18, 19 are respectively CDR1 and CDR2 of the heavy chain of the antibody, and SEQ ID NO: 20, 21, 22 are respectively CDR1 , CDR2 and CDR3 of the antibody, wherein the antibody is produced by the hybridoma cell line 2161 D2; and/or
SEQ ID NO: 23, 24 and SEQ ID NO: 25, 26, 27, wherein SEQ ID NO: 23, 24 are respectively CDR1 and CDR2 of the heavy chain of the antibody, and SEQ ID NO: 25, 26, 27 are respectively CDR1 , CDR2 and CDR3 of the antibody, wherein the antibody is produced by the hybridoma cell line 2164H5; and/or
SEQ ID NO: 28, 29, 30 and SEQ ID NO: 31 , 32, 33, wherein SEQ ID NO: 28, 29, 30 are respectively CDR1 , CDR2 and CDR3 of the heavy chain of the antibody, and SEQ ID NO: 31 , 32, 33 are respectively CDR1 , CDR2 and CDR3 of the antibody, wherein the antibody is produced by the hybridoma cell line 2166G4; and/or
SEQ ID NO: 34, 35, 36 and SEQ ID NO: 37, 38, 39, wherein SEQ ID NO: 34, 35, 36 are respectively CDR1 , CDR2 and CDR3 of the heavy chain of the antibody, and SEQ ID NO: 37, 38, 39 are respectively CDR1 , CDR2 and CDR3 of the antibody, wherein the antibody is produced by the hybridoma cell line 2172B12.

3. The antibody according to claim 1 having SEQ ID NO: 1 as VH and SEQ ID NO: 3 as VL, or SEQ ID NO: 5 as VH and SEQ ID NO: 7 as VL, or SEQ ID NO: 9 as VH and SEQ ID NO: 1 1 as VL, or SEQ ID NO: 13 as VH and SEQ ID NO: 15 as VL.

4. An antibody fragment, or a diabody, or a scFv recognizing and binding to an antigen comprising the ectodomain of the NG2/CSPG4 transmembrane proteoglycan, wherein said antibody induces cancer cell apoptosis and/or autophagy upon high-affinity binding to the antigen said antibody fragment being selected from the group consisting of: half-lgG, Fc, VH, VHH, VL, VLL, F(ab′)2, Fab, Fab′ and Fv.

5. The antibody fragment, or the diabody or the scFv recognizing and binding to an antigen comprising the ectodomain of the NG2/CSPG4 transmembrane proteoglycan, wherein said antibody induces cancer cell apoptosis and/or autophagy upon high-affinity binding to the antigen said antibody fragment being selected from the group consisting of: half-lgG, Fc, VH, VHH, VL, VLL, F(ab′)2, Fab, Fab′ and Fv having the VH and VL according to claim 2.

6. A composition comprising the antibody according to claim 1 or the antibody fragment, or the diabody, or the scFv recognizing and binding to an antigen comprising the ectodomain of the NG2/CSPG4 transmembrane proteoglycan, wherein said antibody induces cancer cell apoptosis and/or autophagy upon high-affinity binding to the antigen said antibody fragment being selected from the group consisting of: half-lgG, Fc, VH, VHH, VL, VLL, F(ab′)2, Fab, Fab′ and Fv and pharmaceutically acceptable excipients.

7. The antibody of claim 1, in its entirety or single chain variable fragment, or scFv, or any other fragmented form that is active against NG2/CSPG4, or any other portion of the antibody, preferably embodied in a bispecific antibody construct or a fusion protein comprised of a therapeutic amount of the antibody, in its entirety or single chain variable fragment, or scFv, and another therapeutic molecule, preferably a cytochine or an aptoptosis-inducing agent, or a CAR T (Chimeric Antigen Receptor T cell) construct in which the scFv derived from the portion of the antibody is expressed on the surface of a CAR T cell, such as to simultaneously engaging NG2/CSPG4 expressing cells and endogenous T cells and favor the maintenance of the CAR T cell in proximity to the cancer cell to trigger the activation of the engaged T cell.

8. Method of treating a disease caused or associated to apoptosis and/or autophagy in an individual in need thereof with the antibody according to claim 1 or with the antibody fragment, or the diabody, or the scFv recognizing and binding to an antigen comprising the ectodomain of the NG2/CSPG4 transmembrane proteoglycan, wherein said antibody induces cancer cell apoptosis and/or autophagy upon high-affinity binding to the antigen said antibody fragment being selected from the group consisting of: half-lgG, Fc, VH, VHH, VL, VLL, F(ab′)2, Fab, Fab′ and Fv and pharmaceutical acceptable excipients, said method comprising
administering to said individual an effective amount of said antibody or said antibody fragment or said diabody or said scFv

9. The method according to claim 8 wherein said disease is cancer, said cancer being selected from the group consisting of: any variant, subtype or histotype of melanomas, soft-tissue, cartilage or bone sarcomas, head or neck carcinomas, colorectal carcinomas, lung carcinomas, prostate carcinomas, breast carcinomas, skin carcinomas, mesotheliomas, hematological neoplasia or Central Nervous System (CNS) and Peripheral Nervous System (PNS) tumors.

10. Method treating and/or preventing secondary metastatic and non-metastatic lesion formation, or relapsing local, loco-regional or distant tumor formation in an individual in need thereof with the antibody according to claim 1 or the antibody fragment, or the diabody, or the scFv recognizing and binding to an antigen comprising the ectodomain of the NG2/CSPG4 transmembrane proteoglycan, wherein said antibody induces cancer cell apoptosis and/or autophagy upon high-affinity binding to the antigen said antibody fragment being selected from the group consisting of: half-lgG, Fc, VH, VHH, VL, VLL, F(ab′)2, Fab, Fab′ and Fv or with the scFv with pharmaceutically acceptable excipients, said method comprising administering to said individual in need thereof an effective amount of said antibody or said antibody fragment or said diabody or said scFv.
